SEQUENCE LISTING (19 sequences)

SEQ ID NO: 1 - RNA, 28 nt, synthetic construct
  source 1..28: mol_type = other RNA
gacagcctgctgtcgacagcctgctgtc

SEQ ID NO: 2 - DNA, 20 bp, synthetic construct
  source 1..20: mol_type = other DNA
cctcatccagtgaggagtcg

SEQ ID NO: 3 - DNA, 20 bp, synthetic construct
  source 1..20: mol_type = other DNA
ccaaagacagactctcagat

SEQ ID NO: 4 - DNA, 20 bp, synthetic construct
  source 1..20: mol_type = other DNA
aaggtgaaggtcggagtcaa

SEQ ID NO: 5 - DNA, 20 bp, synthetic construct
  source 1..20: mol_type = other DNA
attccaaggttggttgaagc

SEQ ID NO: 6 - DNA, 23 bp, synthetic construct
  source 1..23: mol_type = other DNA
aaactggtcatgtagccatcaac

SEQ ID NO: 7 - DNA, 22 bp, synthetic construct
  source 1..22: mol_type = other DNA
tggtgggagaaatgctgtatgc

SEQ ID NO: 8 - DNA, 20 bp, synthetic construct
  source 1..20: mol_type = other DNA
aacatcgccaagggacctgc

SEQ ID NO: 9 - DNA, 20 bp, synthetic construct
  source 1..20: mol_type = other DNA
aatgaaggggtcattgatgg

SEQ ID NO: 10 - DNA, 20 bp, synthetic construct
  source 1..20: mol_type = other DNA
aacccaaaccaatgaagctg

SEQ ID NO: 11 - DNA, 21 bp, synthetic construct
  source 1..21: mol_type = other DNA
aattgtgcattcccgtgcaag

SEQ ID NO: 12 - RNA, 14 nt, synthetic construct
  source 1..14: mol_type = other RNA
gacagcctgctgtc

SEQ ID NO: 13 - RNA, 54 nt, synthetic construct
  source 1..54: mol_type = other RNA
gcgcctgcctgcctgcctgcctgcctgcctgcctgcctgcctgcctgcctgcgc

SEQ ID NO: 14 - RNA, 54 nt, synthetic construct
  source 1..54: mol_type = other RNA
gcgcctgcctgcctgcctgcctgcctgcctggcaggcaggcaggcaggcagcgc

SEQ ID NO: 15 - RNA, 48 nt, synthetic construct
  source 1..48: mol_type = other RNA
cctgcctgcctgcctgcctgcctgcctgcctgcctgcctgcctgcctg

SEQ ID NO: 16 - RNA, 40 nt, synthetic construct
  source 1..40: mol_type = other RNA
cctgcctgcctgcctgcctgcctgcctgcctgcctgcctg

SEQ ID NO: 17 - RNA, 46 nt, synthetic construct
  source 1..46: mol_type = other RNA
gcgcctgcctgcctgcctgcctgcctgcctgcctgcctgcctgcgc

SEQ ID NO: 18 - RNA, 46 nt, synthetic construct
  source 1..46: mol_type = other RNA
gcgcctgcctgcctgcctgcctgcctgcaggcaggcaggcaggcgc

SEQ ID NO: 19 - RNA, 14 nt, synthetic construct
  source 1..14: mol_type = other RNA
gacagcctgctgtc

(19) United States
(12) Patent Application Publication
(10) Pub. No.: US 2024/0262817 A1
(43) Pub. Date: Aug. 8, 2024

(54) QUINAZOLINE DERIVATIVES TARGETING R(CCUG) REPEATS IN MYOTONIC DYSTROPHY TYPE 2

(71) Applicant: University of Florida Research Foundation, Incorporated, Gainesville, FL (US)

(72) Inventor: Matthew D. Disney, Gainesville, FL (US)

(73) Assignee: University of Florida Research Foundation, Incorporated, Gainesville, FL (US)

(21) Appl. No.: 18/560,558

(22) PCT Filed: Jul. 13, 2022
(86) PCT No.: PCT/US2022/073706

§ 371 Date: Nov. 13, 2023

Related U.S. Application Data
(60) Provisional application No. 63/188,884, filed on May 14, 2021.

(51) Int. Cl. C07D 417/12; A61K 31/517; A61K 31/5377; A61P 21/00; C07D 403/12; C07D 413/12; C07D 417/14

(52) U.S. Cl. CPC C07D 417/12; A61K 31/517; A61K 31/5377; A61P 21/00; C07D 403/12; C07D 413/12; C07D 417/14

(57) ABSTRACT

A group of quinazoline compounds has been developed that interrupt the r(CCUG)^{exp-}MBNL1 complex and lessen pre-mRNA splicing errors due to sequestered activation and inhibition regulator MBNL1. A method of treatment of myotonic dystrophy type 2 using these quinazoline compounds has been developed.

### STATEMENT OF GOVERNMENT SUPPORT

This invention was made with government support under grant number NS116846 awarded by the National Institutes of Health. The government has certain rights in the invention.

### REFERENCE TO AN ELECTRONIC SEQUENCE LISTING

The contents of the electronic sequence listing (U120270098WO00-SEQ-JDH.xml; Size: 17,992 bytes; and Date of Creation: Jul. 13, 2022) is herein incorporated by reference in its entirety.

### BACKGROUND

RNA repeat expansions cause >40 microsatellite disorders, including Huntington's disease [HD, r(CAG)^{exp}], c9ORF72 amyotrophic lateral sclerosis/frontotemporal dementia [ALS/FTD, r(G₄C₂)^{exp}], and myotonic dystrophy types 1 and 2 [DM1, r(CUG)^{exp} and DM2. r(CCUG)^{exp}, respectively].¹ Repeat expansions cause disease by different mechanisms, dependent upon its location within a gene. For example, an RNA gain-of-function mechanism occurs when the repeat sequesters and functionally inactivates proteins that regulate alternative pre-mRNA splicing.² Repeat expansions can also contribute to disease via an aberrant translational mechanisms that generates toxic proteins named repeat associated non-ATG translation.³ Recently, it was discovered that GC-rich RNA repeat expansions harbored in introns, such as r(CCUG)^{exp}. r(CUG)^{exp}, and r(G₄C₂)^{exp}, cause retention of the intron in which they are harbored in mature mRNA species.⁴

The DM2 repeat expansion, r(CCUG)^{exp}, is harbored in intron 1 of CCHC-Type Zinc Finger Nucleic Acid Binding Protein gene (CNBP).⁵ This r(CCUG)^{exp} folds into a structure containing repeating 2×2 CU/UC internal loops (FIG. 1A).⁶ This repeating CCUG structure binds and sequesters muscleblind-like 1 (MBNL1), an important regulator of alternative pre-mRNA splicing, in nuclear foci.^{7, 8} Sequestration of MBNL1 by r(CCUG)^{exp} results in pre-mRNA splicing defects of MBNL1-regulated genes, for example insulin receptor (IR) exon 11 which is excluded too frequently in DM2 cells (FIG. 1B).⁹ The r(CCUG)^{exp} can also cause dysfunction through intron retention where CNBP itself is mis-spliced, and the r(CCUG)^{exp}-containing intron 1 is retained in mature mRNA species (FIG. 1C).⁴

It has been previously shown that intron retention is due to MBNL1 binding; knock-down of MBNL1 reduces intron retention whereas transfection of MBNL1 increases intron retenion.¹⁰ Thus, small molecule recognition of r(CCUG)^{exp} liberates MBNL1 and rescues deregulated splicing events and intron retention. In particular, it has been shown that kanamycin A derivatives that bind r(CCUG)^{exp} can specifically improve DM2-associated defects and reduce the levels of intron 1 containing r(CCUG)^{exp}.¹⁰ In another example of an intron-retained RNA repeat [r(CUG)^{exp} in intron 3 of transcription factor 4 mRNA (TCF4) in Fuchs endothelial corneal dystrophy (FECD)],¹¹ small molecule binding results in decay of the r(CUG)^{exp}-containing intron 3 through the RNA exosome.¹² Thus, small molecule recognition of RNA repeat expansions in retained introns can allow for targeted RNA degradation through stimulation of RNA quality control mechanisms. In addition to interfacing of disease-causing RNAs with endogenous decay pathways, other methods for targeted RNA degradation rely on chimeric compounds comprising an RNA-binding ligand attached to a cleaving module. However, this strategy increases compound molecular weight and can affect various pharmacological properties. 13-17

Although previous studies targeting r(CCUG)^{exp} showed that small molecules can indeed interface the toxic RNA with endogenous degradation pathways, the aminoglycosides from which they are derived are not particularly drug-like and provide little opportunity for optimization.^{10, 18} Therefore, an object according to the invention is to develop small molecules that target r(CCUG)^{exp} and can function as effective drugs for treatment of r(CCUG)^{exp} related disease.

### SUMMARY

Aspects of the present invention are directed to novel compounds that bind r(CCUG)^{exp} and improve DM2-associated defects in patient-derived cells by high-throughput screening of a drug-like RNA-focused small molecule library. The physiochemical properties of the compounds identified herein indicate that drug-like small molecules can indeed selectively bind RNA structures. The ability to selectively degrade introns containing RNA repeat expansions with drug-like small molecules will have broad implications in drug discovery efforts towards targeting RNAs.

These and other objects are achieved by compositional, assay, and method of treatment aspects of the present invention. Beginning with a study of a collection of RNA-focused, drug-like small molecules to target r(CCUG)^{exp}, screening identified a group of small molecules that effectively bind r(CCUG)^{exp}. A medicinal chemistry research program applied to the screening hits afforded a group of novel drug-like small molecules that bind r(CCUG)^{exp} with nanomolar affinity. The medicinal chemistry research in particular afforded a quinazoline core compound with 20-fold improvement in vitro activity and 5-fold improvement in cellular activity. This novel small molecule was shown to have strong binding with r(CCUG)^{exp}, to lack binding with other RNAs, have very low toxicity, specifically degrade r(CCUG)^{exp}-containing intron 1, and improve DM2 defects. This quinazoline core compound selectively improves various DM2-associated defects including intron retention of r(CCUG)^{exp}-containing intron 1, which is decayed in cells upon treatment with the compound.

Accordingly, one aspect of the invention is directed to compositional embodiments of the invention. These compositional embodiments comprise a quinazoline compound of Formula I

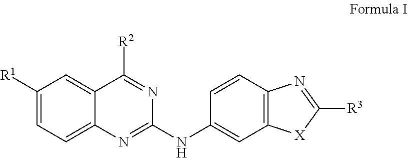

Formula I includes the following substituents:
R¹ is hydrogen or alkyl of 1 to 3 carbons;
R² is —NH(CH₂)ₙOH with n being an integer of 2, 3 or 4
R³ is hydrogen, alkyl of 1 to 3 carbons, —NH(CH₂)C(R⁴)₂OH with each instance of R⁴ independently being hydrogen or methyl or —NH(CH₂)₂ NMe₂;
X is oxygen, sulfur or NH.
Preferably, X is sulfur and R¹ is alkyl, preferably methyl.
Preferably R² has n as 3.
Preferably R³ methyl or —NH(CH₂)C(CH₃)₂OH.

An especially preferred embodiment of Formula I is the quinazoline core compound of Formula I with X as sulfur, R¹ as methyl, R² as —NH(CH₂)₃OH, and R³ as methyl. This especially preferred embodiment can form a complex with r(CCUG)^{exp} with a K_{d} in the range of 170-220 nM.

A compositional aspect of the present invention is also directed to a combination of a quinazoline compound of Formula I in combination with an r(CCUG)₁₂-MBNL1 complex.

Aspects of the present invention are also directed to a method for assaying the ability to interrupt r(CCUG)^{exp}-MBNL1 complex by contacting a compound of Formula I with DM2 patient derived fibroblasts carrying the complex and observing a reduction of the number of complexes by assaying for uncomplexed MBNL1. The presence of uncomplexed MBNL1 can be determined by RNA fluorescence in situ hybridization (FISH) and immunofluorescence.

Further aspects of the present invention are directed to reduction of the r(CCUG)^{exp}-MBNL1 complex and the intron harboring the repeat expansion, thereby leading to improvement of aberrant alternative pre-mRNA splicing in myotonic dystrophy type 2 cells comprising contacting a tissue sample of a DM2 patient carrying DM type 2 cells with a composition of Formula I or with a pharmaceutical composition comprising a composition of Formula I and a pharmaceutically acceptable carrier.

Additional aspects of the present invention are directed to treatment of a patient suffering from myotonic dystrophy type 2 comprising administering to the patient an effective amount of a composition of Formula I or a pharmaceutical composition comprising an effective amount of a composition of Formula I and a pharmaceutically acceptable carrier.

An additional aspect of the invention is directed to a combination of an r(CCUG)₁₂-MBNL1 complex disruption by the small molecule or the binding of the small molecule to a single 2×2 CU/UC RNA to study its avidity, here a second quinazoline compound of Formula II. The second quinazoline compound constitutes the lead hits of the screening and their combination of the complex or single RNA provides a novel combination indicating a strong binding constant thereby showing an ability to function to interrupt the r(CCUG)^{exp}-MBNL1 complex. Formula II of the second quinazoline compound comprises:

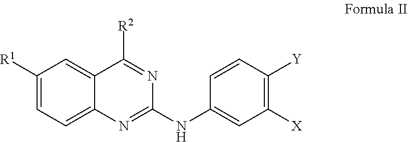

The substituents of Formula II include:
R¹ as hydrogen or alkyl of 1 to 3 carbons;
R² as —NH(CH₂)ₙOH with n being an integer of 2, 3 or 4, —NHCH₂-(2-tetrahydrofuranyl);
Y as —OMe, —NMe₂, —NHAc and X is H or Y and X together as —O—CH₂—CH₂—O—.

A preferred embodiment of the second quinazoline compound of Formula II provides R¹ as hydrogen or methyl, R² as NH(CH₂)₃OH and Y and X together are —O—CH₂—CH₂—O—.

The foregoing method for assaying the ability to interrupt r(CCUG)^{exp}-MBNL1 complexation may also be conducted with a second quinazoline compound of Formula II.

### BRIEF DESCRIPTION OF DRAWINGS

FIG. 1A, 1B, 1C depicts r(CCUG)^{exp}-mediated defects in DM2.

FIG. 1A shows that DM2 is caused by r(CCUG)^{exp} in CNBP intron 1, which folds into a structure with repeating 2×2 internal loops that sequester regulatory proteins involved in pre-mRNA splicing such as MBNL1.

FIG. 1B shows that (B) Sequestration of MBNL1 by r(CCUG)^{exp} results in splicing defects in its native pre-mRNAs substrates. For example, IR, exon 11 is included ˜70% of the time in healthy cells, but only ˜50% in DM2 cells.

FIG. 1C shows that the r(CCUG)^{exp} also causes intron retention where intron 1 is aberrantly retained in CNBP mature RNA.

FIG. 2A, 2B, 2C, 2D depicts small molecules that inhibit r(CCUG)^{exp}-MBNL1 complex formation in vitro.

FIG. 2A shows characteristics of an RNA-focused small molecule library compared to characteristics of known drugs in DrugBank.

FIG. 2B shows IC₅₀ values of hit compounds from RNA-focused library screen for disrupting an r(CCUG)₁₂-MBNL1 complex (n=2). Molecules in red have IC₅₀s <50 μM.

FIG. 2C shows the Chemical structures of compounds with IC₅₀s <50 μM. FIG. 2D shows rescue of the IR exon 11 splicing defect by 3, 4, 5, and 7 in DM2 fibroblasts (n=2). Error bars represent standard deviation (SD) for all panels.

FIG. 3A, 3B, 3C, 3D, 3E show lead optimization of 7 and activity of derivatives in vitro and in DM2 patient-derived fibroblasts.

FIG. 3A shows structures of derivatives of 7 where “R” indicates the position(s) that were varied. IC₅₀ values for disrupting an r(CCUG)₁₂-MBNL1 complex for each derivative (n=3).

FIG. 3B shows structures of compounds with IC₅₀s <5 μM.

FIG. 3C shows rescue of IR exon 11 mis-splicing by non-toxic compounds 63, 79, and 80 in DM2 fibroblasts (n=3). ** P<0.01. *** P<0.001, as determined by a one-way ANOVA relative to untreated (“0”).

FIG. 3D shows binding affinity curve of 63 and r(CCUG)₁₂ (SEQ ID NO: 15)(K_{d} 192±20 nM).

FIG. 3E shows the binding affinity curve of 63 and a base pair control RNA (K_{d}>20 μM). Error bars represent standard deviation (SD) for all panels.

FIG. 4A, 4B, 4C, 4D shows that compound 63 rescues disease-associated defects in DM2 patient-derived fibroblasts.

FIG. 4A shows representative RNA FISH and MBNL1 immunofluorescence images of r(CCUG)^{exp}-MBNL1 foci.

FIG. 4B shows quantification of the number of nuclear foci/cell (n=3, 40 nuclei counted/replicate). *** P<0.001, as determined by a Student t-test.

FIG. 4C shows analysis of CNBP intron 1 levels upon treatment with 63 via RT-qPCR (n=3). * P<0.05. *** P<0.001, as determined by a one-way ANOVA.

FIG. 4D shows analysis of CNBP mature mRNA levels upon treatment with 63 via RT-qPCR (n=3). Error bars represent standard deviation (SD) for all panels.

FIG. 5 depicts a schematic showing how a quinazoline embodiment of the invention targets RNA repetition and facilitates its degradation.

FIG. 6. Results of a high throughput screen to identify small molecules that disrupt the r(CCUG)₁₂-MBNL1 complex, a validated in vitro model¹ of the disease-causing interaction that causes DM2. All 3271 compounds in the library were screened at 200 μM. Compounds that exhibited a percent disruption >3 standard deviations from the mean (3s; >60% disruption) are highlighted in green.

FIG. 7. Structures of compounds 1-44. Structures of hit compounds from the high throughput screen of the RNA-focused library that disrupt r(CCUG)₁₂-MBNL1 complex greater than 60%.

FIGS. 8A 8B show WaterLOGSY NMR analysis of 3 with SEQ ID NO:1 r(GACAGCCUGCUGUC)₂ duplex.

FIG. 8A is the 1D ¹H NMR of 3.

FIG. 8B is the WaterLOGSY NMR spectra of 3.

FIGS. 9A and 9B show WaterLOGSY NMR analysis of 4 with SEQ ID NO:1 r(GACAGCCUGCUGUC)₂ duplex.

FIG. 9A shows the 1D ¹H NMR of 4.

FIG. 9B shows the WaterLOGSY NMR spectra of 4.

FIGS. 10A and 10B show the WaterLOGSY NMR analysis of 5 with SEQ ID NO:1 r(GACAGCCUGCUGUC)₂ duplex.

FIG. 10A shows the 1D ¹H NMR of 5. FIG. 10B shows the WaterLOGSY NMR spectra of 5.

FIGS. 11A and 11B show the WaterLOGSY NMR analysis of 7 with SEQ ID NO:1 r(GACAGCCUGCUGUC)₂ duplex.

FIG. 11A shows the 1D ¹H NMR of 7.

FIG. 11B shows the WaterLOGSY NMR spectra of 7.

FIG. 12 shows the 2D NMR analysis of SEQ ID NO: 1 r(GACAGCCUGCUGUC)₂ duplex in the absence of compound. 2D NOE spectra for RNA assignments.

FIG. 13 shows the 1D NMR analysis of compounds bound to SEQ ID NO:1 r(GACAGCCUGCUGUC)₂. 1D spectra of unbound RNA (bottom) and shifts in spectra upon addition of compound.

FIGS. 14A-14D shows the cell viability of DM2 fibroblasts treated with 3, 4, 5, and 7 assessed via CellTiter-Glo. Viability of cells treated with 3 (FIG. 14A), 4 (FIG. 14B), 5 (FIG. 14C), and 7 (FIG. 14D). For all panels, n=5 and error bars represent SD.

FIG. 15 shows the structures of compounds 45-82.

FIGS. 16A-16H show the cell viability of DM2 fibroblasts treated with lead optimized compounds assessed via Cell-Titer Glo. For all panels, n=5 and error bars represent SD. The viability of cells treated with the quinazoline compounds is shown by the graphs of

FIG. 16A, quinazoline compound 63;

FIG. 16B quinazoline compound 71;

FIG. 16C quinazoline compound 72;

FIG. 16D quinazoline compound 73;

FIG. 16E quinazoline compound 74;

FIG. 16F quinazoline compound 78;

FIG. 16G quinazoline compound 79;

FIG. 16H quinazoline compound 82.

FIGS. 17A, 17B, 17C, 17D, 17E show the cellular activity of compounds 63, 78, and 79 in DM2 fibroblasts, as assessed by rescue of IR exon 11 mis-splicing.

FIG. 17A shows a representative gel image of IR exon 11 splicing in DM2 fibroblasts treated with 63.

FIG. 17B shows quantification of rescue of IR exon 11 splicing.

FIG. 17C shows representative gel image of IR exon 11 splicing in DM2 fibroblasts treated with 78 or 79.

FIG. 17D shows quantification of rescue of IR exon 11 splicing in DM2 fibroblasts treated with 78.

FIG. 17E shows quantification of rescue of IR exon 11 splicing in DM2 fibroblasts treated with 79. For all panels, n=3 and error bars represent SD; ** P<0.01, *** P<0.001, as determined by one-way ANOVA relative to untreated.

FIGS. 18A, 18B show that compound 63 does not affect MAP4K4 (non-MBNL1 regulated) splicing in DM2 fibroblasts.

FIG. 18A shows a representative gel image of MAP4K4 exon 22a splicing in DM2 fibroblasts treated with 63.

FIG. 18B shows quantification of MAP4K4 exon 22a inclusion; n=3; error bars represent SD.

FIGS. 19A, 19B, 19C show that compound 63 has no effect on CNBP levels or IR exon 11 splicing in wild-type fibroblasts.

FIG. 19A shows the analysis of CNBP intron 1 levels in wild-type fibroblasts treated with 63 via RT-qPCR.

FIG. 19B shows a representative gel image of IR exon 11 splicing in wild-type fibroblasts treated with 63.

FIG. 19C shows quantification of IR exon 11 inclusion. For all panel: n=3 and error bars represent SD.

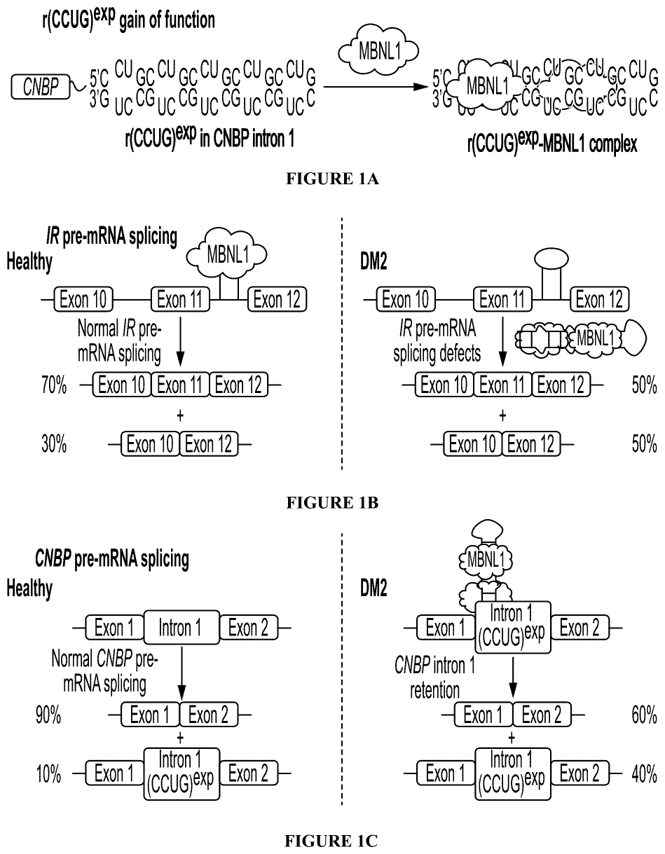

### DEFINITIONS

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The term and/or means both as well as one or the other as in A and/or B means A alone, B alone and A and B together

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

At various places in the present specification substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number.

Standard abbreviations for chemical groups such as are well known in the art are used; e.g., Mc=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, ß-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

Each of the terms “halogen,” “halide,” and “halo” refers to —F, —Cl, —Br, or —I.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stercomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

The terms “prevent.” “preventing,” and “prevention” refer to the prevention of the onset, recurrence, or spread of the disease in a patient resulting from the administration of a prophylactic or therapeutic agent.

A “patient” or “subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

### DETAILED DESCRIPTION

Study of an RNA focused small molecule compound collection for targeting r(CCUG).

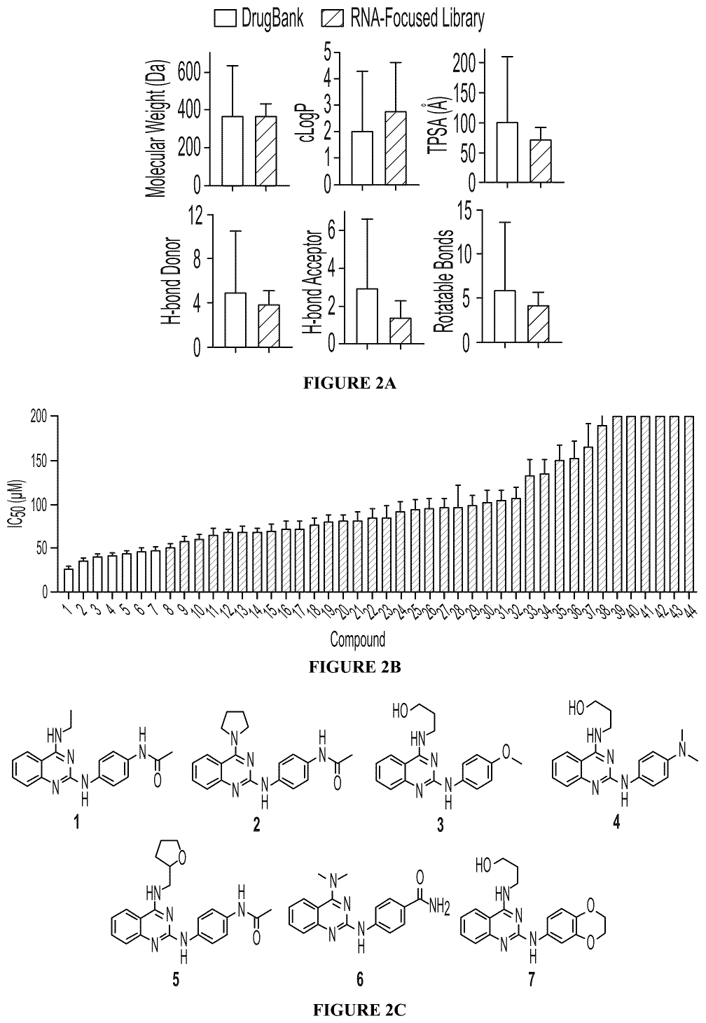

To identify novel small molecules that bind r(CCUG)^{exp}, a drug-like RNA focused small molecule library containing 3,271 compounds was employed.¹⁹ This library was previously designed to contain structurally diverse small molecules that have chemotypes that confer avidity towards RNA binding.¹⁹ These chemotypes include benzimidazoles, indoles, thiazoles, and quinazolines. Furthermore, the compounds in this library are drug-like as defined by satisfying Lipinksi's Rule of 5²⁰ where cLogP is <5, molecular weight is <500, the number of hydrogen bond donors is <5, and the number of hydrogen bond acceptors is <10 (FIG. 2A). Historically, drug-likeness has been defined by satisfying the Rule of 5, however an increasing number of approved orally bioavailable drugs fall outside of the Rule of 5, and parameters such as molecular weight are increasing.^{21,22} On average, the library also contains similar physiochemical properties to known drugs in DrugBank (FIG. 2A).¹⁹ Thus, this library can be used to identify drug-like small molecules that bind to RNA structures.

To identify compounds that bind r(CCUG)^{exp}, a previously reported time-resolved fluorescence resonance energy transfer (TR-FRET) assay was used that measures disruption a pre-formed r(CCUG)₁₂-MBNL1 complex by small molecules or other modalities.^{23, 24} The 3,271 member library was screened at 200 μM, and 44 displayed >60% disruption, which corresponds to 3 standard deviations from the mean (or 3s), affording a hit rate of 1.34% (Figure S1). Of the 44 hits, 38 contained a similar substituted N2-phenylquinazoline-2,4-diamine core, while the other hits contained benzimidazoles and indazoles (Figure S2). Importantly, these 44 compounds are drug-like as they have similar physicochemical properties to the rest of the library and to known-drugs in Drug Bank (FIG. 2A). On average, the 44 hit compounds have a molecular weight of 327±35 g/mol, a cLogP of 3.2±0.9. TPSA of 76±15 Å, and 2.5±0.9 hydrogen bond donors. The in vitro potencies (IC₅₀s) of the 44 compounds were then measured in the TR-FRET assay, affording seven compounds (1-7) with IC₅₀s <50 μM (highlighted in red in FIGS. 2B & 2C). Interestingly, all seven of these compounds contained the substituted N2-phenylquinazoline-2,4-diamine core.

### Analysis of Hit Compounds

The binding 1-7 an RNA containing a single 2×2 CU/UC was further studied by NMR spectroscopy. Compounds 1, 2, and 6 showed significant aggregation in NMR experiments and were excluded from further analysis. In contrast, waterLOGSY spectra showed that 3, 4, 5, and 7 bound to the RNA, based on positive phase signals in the presence of RNA (FIGS. S3-S6). To confirm these interactions, 1D NMR spectra of 3, 4, 5, and 7 bound to the RNA containing one 2×2 CU/UC internal loop were analyzed. Aromatic protons of the unbound RNA were assigned via a 2D NOESY spectrum (Figure S7). Significant shifts in resonances corresponding to H6 and H8 protons of the RNA were observed in the presence of these four compounds, indicating binding to the RNA (Figure S8).

After confirming binding to r(CCUG) repeats by NMR spectroscopy, the compounds were evaluated for their ability to improve the deregulation of the IR exon 11 alternative splicing in DM2 patient-derived fibroblasts. All four compounds were well tolerated in the patient-derived fibroblasts, as no significant toxicity was observed up to a 20 UM dose (Figure S9). As aforementioned, IR exon 11 is excluded too frequently In DM2 patient-derived fibroblasts as compared to wild-type cells. Interestingly, the most potent compound, 7, rescued splicing by ˜50% at 5 μM (FIG. 2D).

### Synthesis and In Vitro Analysis of Derivatives of 7.

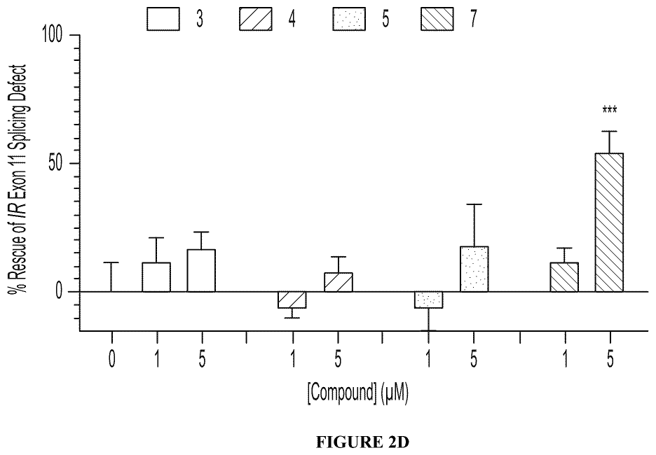

To improve the potency of 7, a library of derivatives that alter the functional groups on the N2-phenylquinazoline-2,4-diamine core was synthesized and then evaluated for disrupting the r(CCUG)₁₂-MBNL1 complex in vitro. The library was synthesized with variations about the N2-phenyl substituent and the quinazoline (Figure S10). Compounds were synthesized starting from 2,4-dichloroquinazoline or a 2,4-dichloropyrimide derivate, followed by the addition of the 3-aminopropan-1-ol linker. Finally, the N2 substituent was installed with the use of various amine-containing heterocycles (Scheme 1 in Supporting Information). Of the 9 compounds in which the N2-2,3-dihydrobenzo[b][1,4]dioxine was exchanged for various heterocycles, three derivatives containing 2-methylbenzo[d]oxazole (49), 2-methylbenzo[d]thiazole (50), and 1-methyl-1H-benzo[d]imidazole (51) substituents had IC₅₀s that were ˜ 2-8 times lower than 7 (IC₅₀=48.2±0.07 μM) (FIG. 3A). Eight derivatives with variations about the quinazoline were then evaluated, and one compound containing 6-methylquinazoline (58) had an IC₅₀ of 12.7±0.2 μM, about four times more potent than 7 (FIG. 3A).

To combine features of the derivates with improved IC₅₀s, three additional compounds were synthesized that contained 6-methylquinazoline and N2-2-methylbenzo[d]oxazole (62), 2-methylbenzo[d]thiazole (63), and 1-methyl-1H-benzo[d]imidazole (64) (FIG. 3A). Compounds 62 and 63, had slightly lower IC₅₀s than their unmethylated derivates (49 and 50, respectively), and 63 was the most potent compound synthesized thus far with an IC₅₀ of 2.2±0.5 μM (FIG. 3A).

To investigate if the addition of extended functional groups increases the in vitro activity of 63, a library of 18 derivates with extensions built onto benzothiazole were synthesized (Schemes 2 and 3 and Figure S10). Six of these derivatives were synthesized via Suzuki coupling using various substituted aryl boronic acids and a derivate of 63 containing a bromo-benzothiazole (Intermediate D, Scheme 2). The other 12 derivates were synthesized via reaction of various amines with the bromo-benzothiazole derivative of 63 (Intermediate D, Scheme 3). Seven compounds (71, 72, 73, 74, 78, 79, and 82; FIG. 3A) had IC₅₀s <5 μM and were chosen to investigate further, along with 63 (FIG. 3B). Interestingly, four compounds (71-74) had IC₅₀s <500 nM which represents a 100-fold improvement in in vitro potency compared to the starting compound, 7.

### Evaluation of Cellular Activity of Compounds.

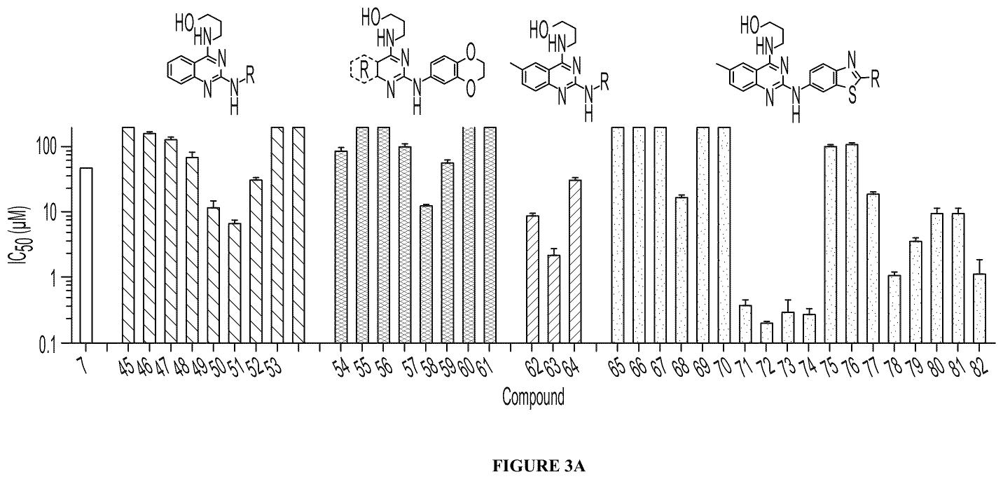

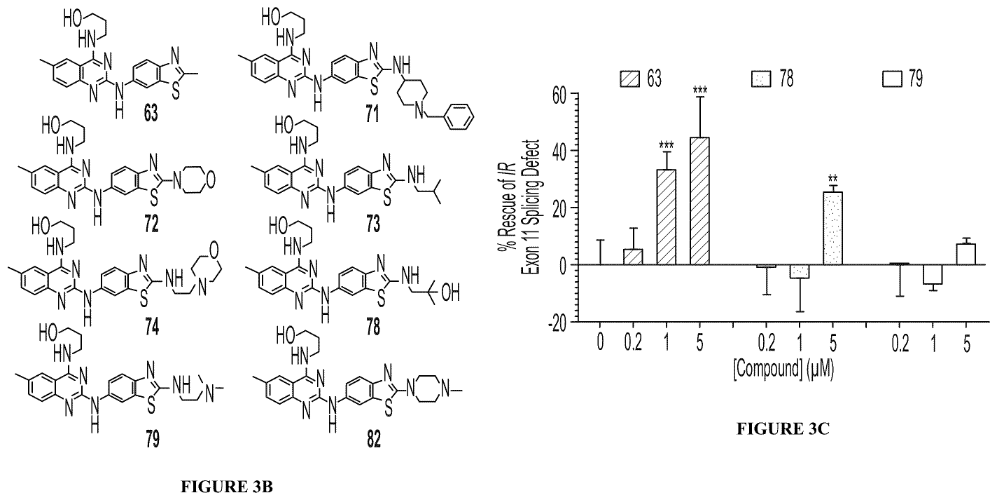

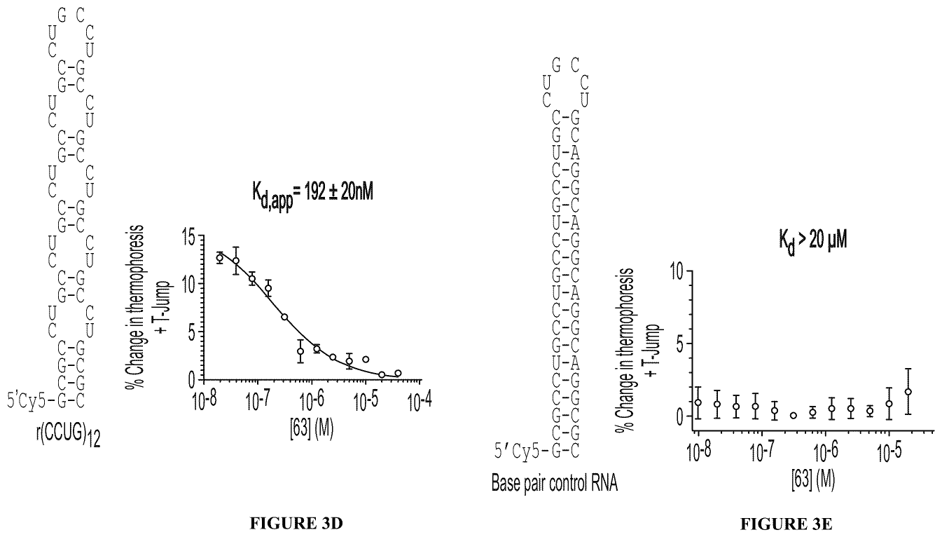

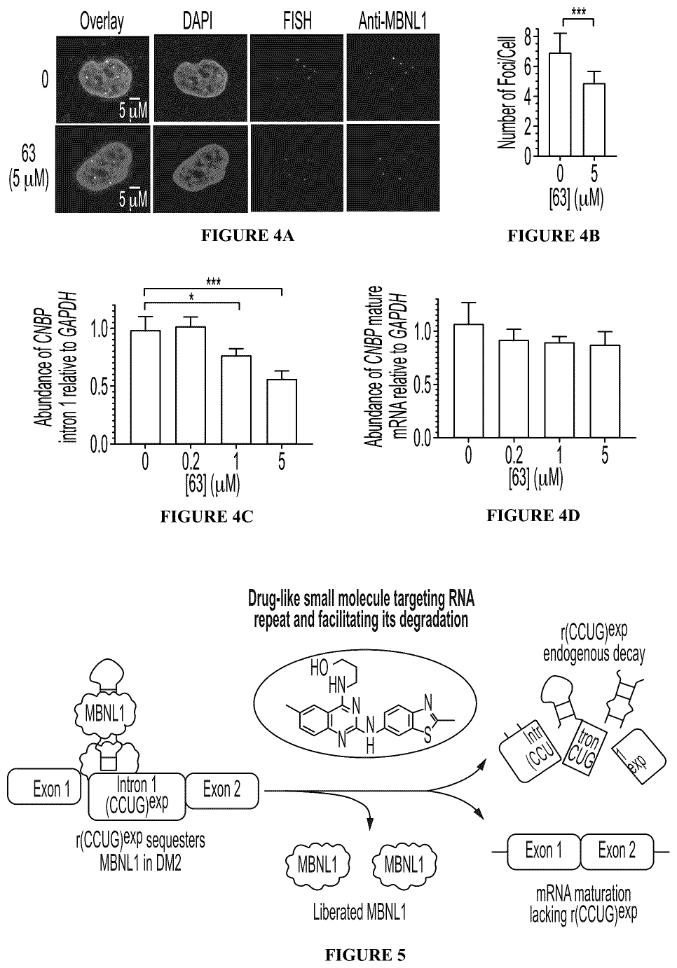

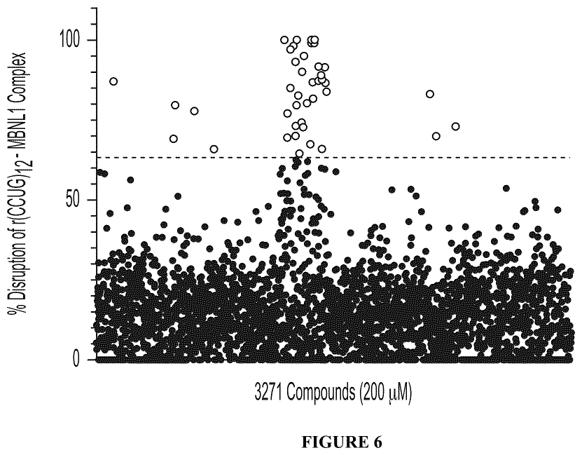

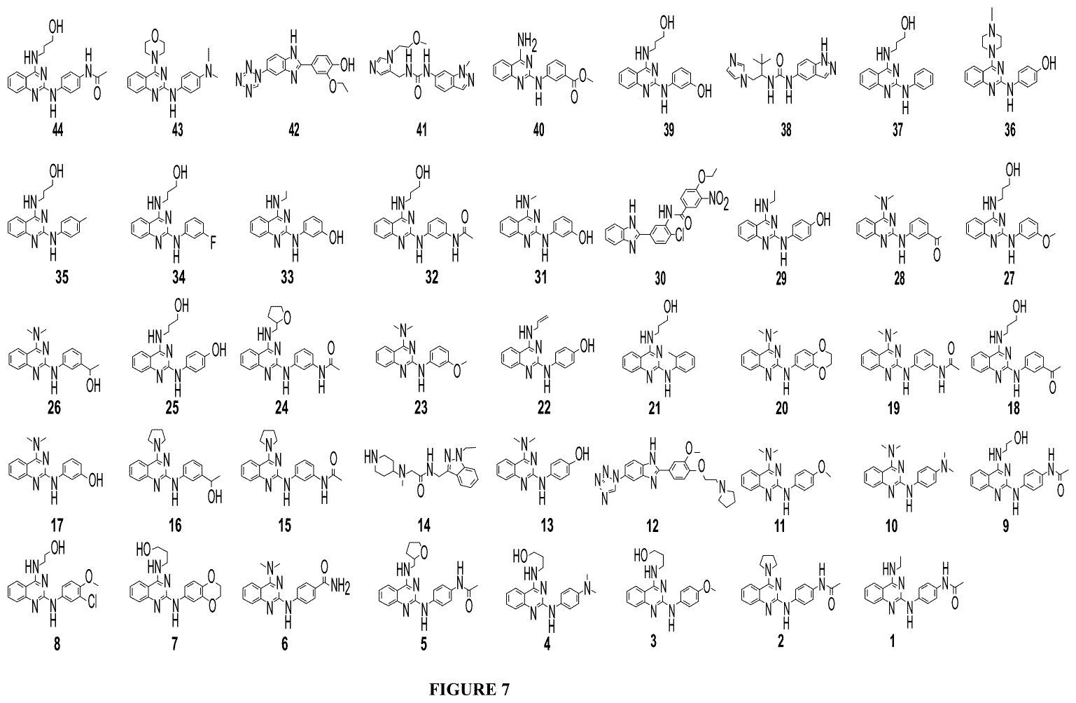

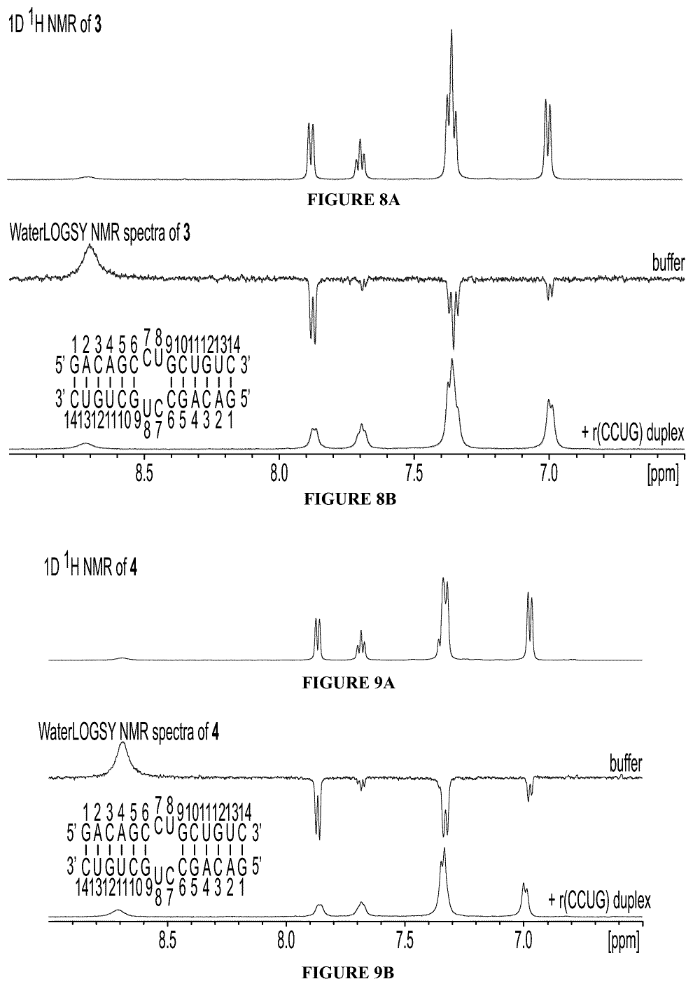

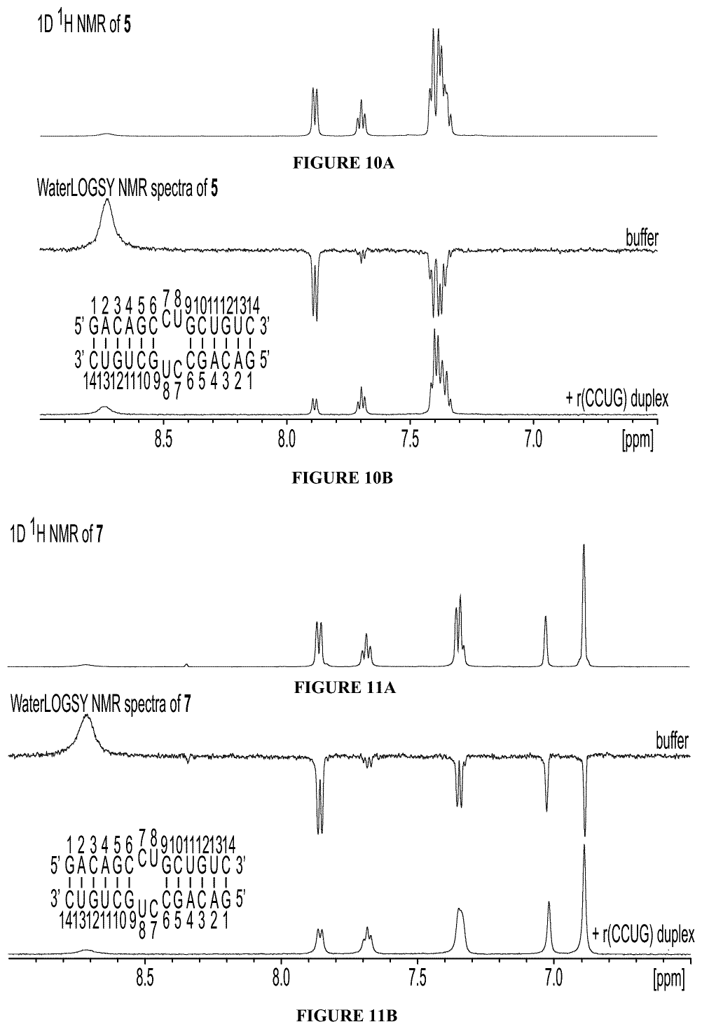

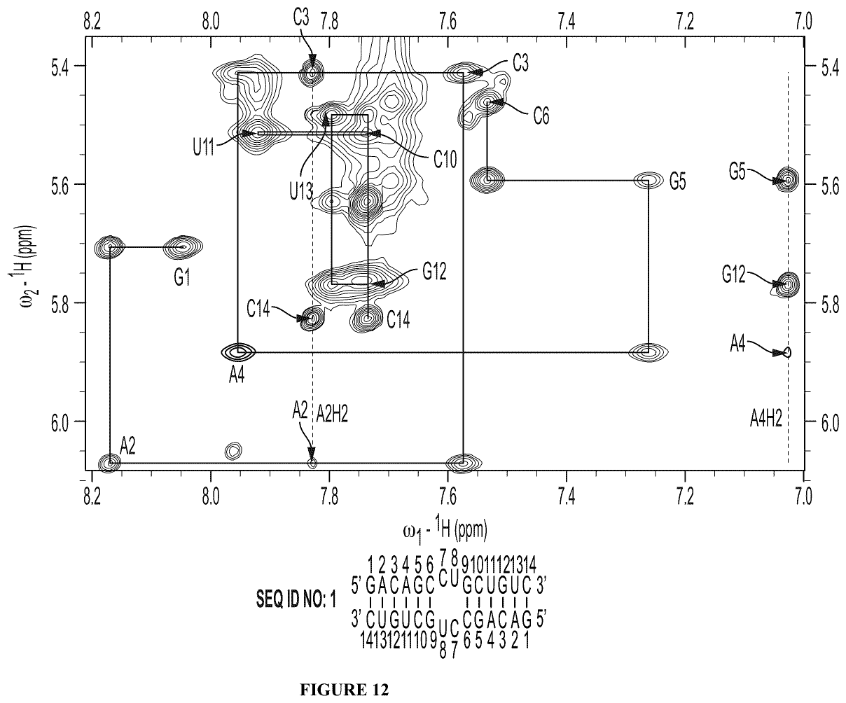

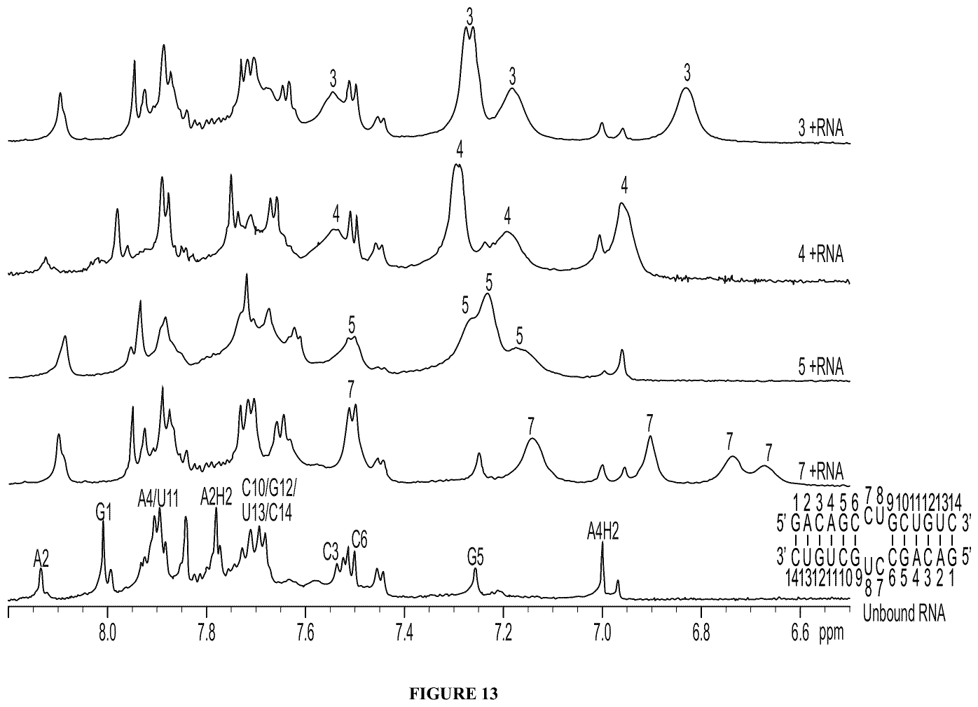

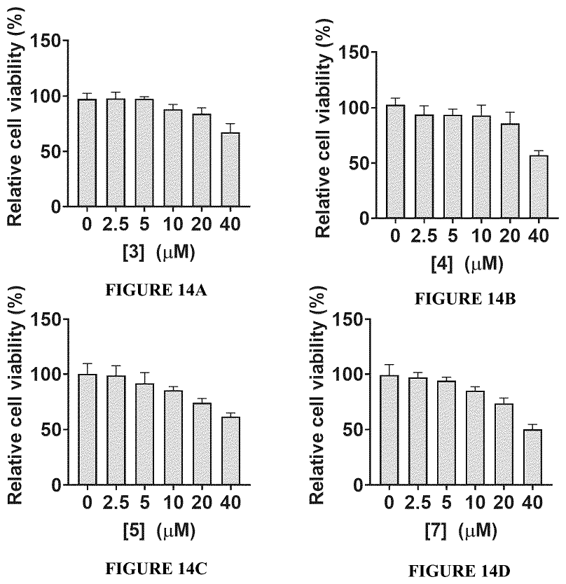

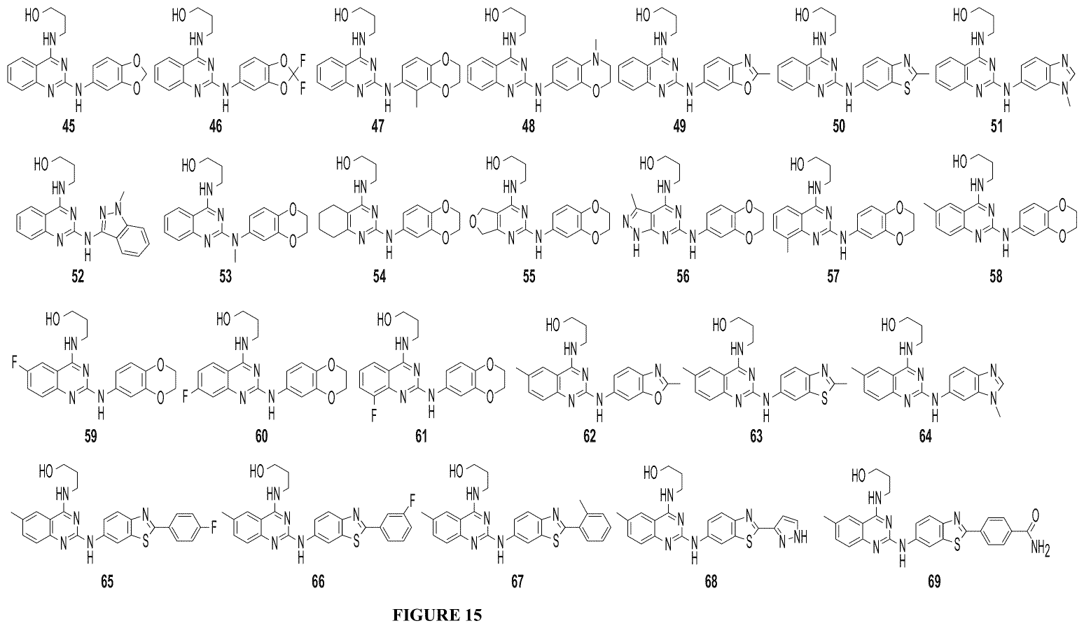

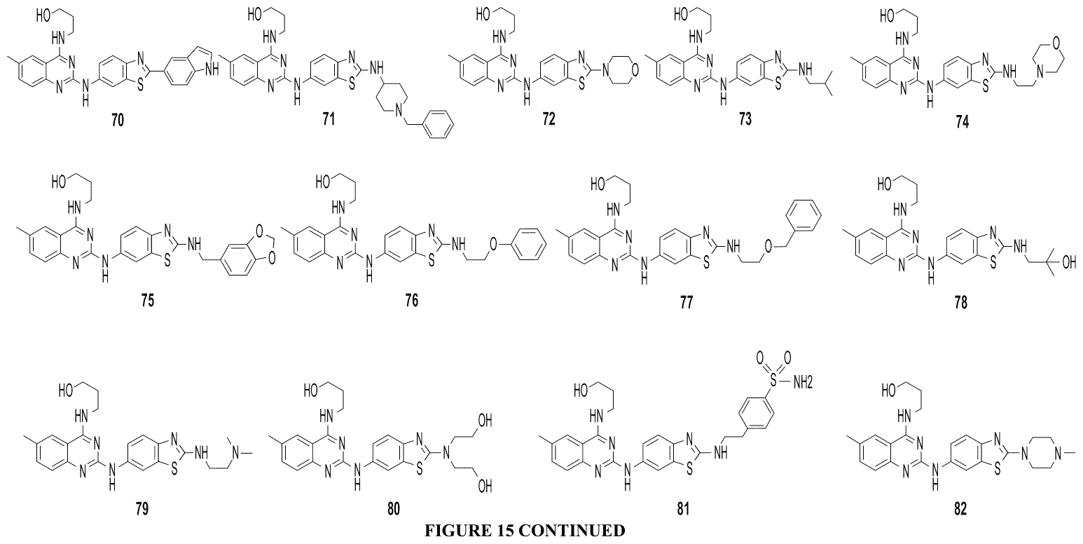

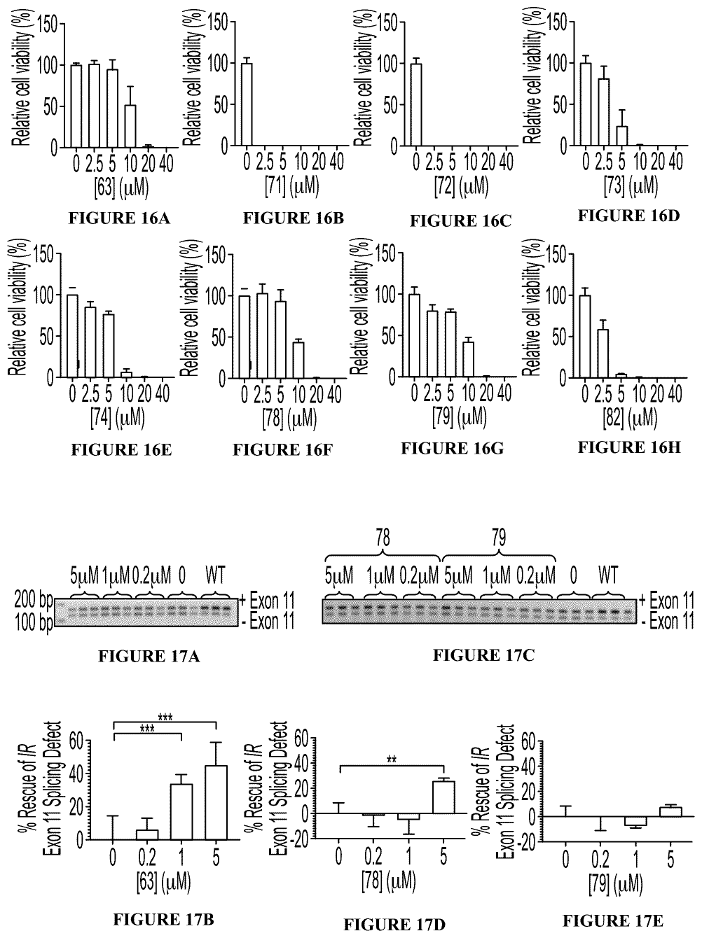

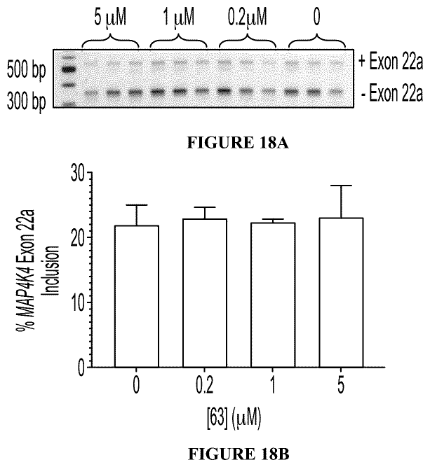

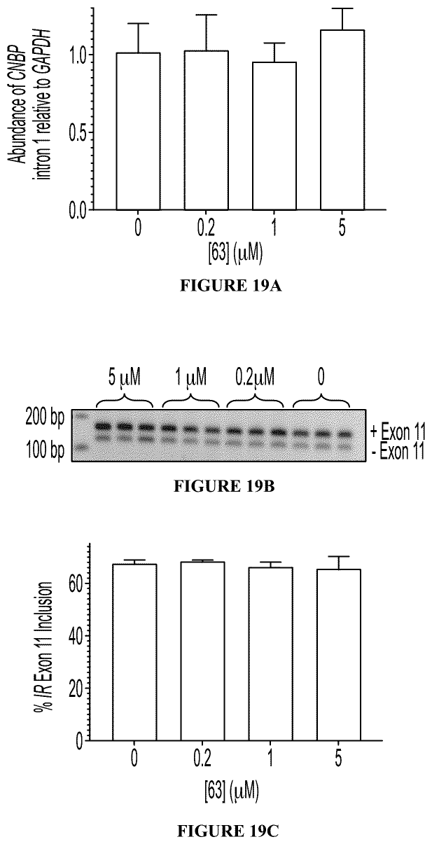

The eight compounds with in vitro IC₅₀s <5 μM were further studied in DM2 patient-derived fibroblasts, both for the effect on cell viability and rescue of IR exon 11 splicing. Compounds 71, 72, 73, 74, and 82 displayed significant toxicity at 5 μM (Figure S11) and were not further evaluated. Of compounds with no toxicity at 5 μM (63, 78, and 79), 63 and 78 significantly improved IR splicing at 5 μM, while 63 also rescued splicing, by ˜30%, at 1 μM (FIG. 3D and S12). Compound 79 did not significantly rescue splicing at any concentration studied (FIG. 4D and S12). At 1 μM dose, 63 rescues IR splicing more potently than 7 (33±5 vs. 12±5; p=0.026), and thus 63 is the most potent compound identified in these studies for improving DM2-associated splicing defects in cells.

The affinity and specificity of 63 for r(CCUG)^{exp} was measured by microscale thermophoresis (MST). Compound 63 binds to r(CCUG)₁₂ (SEQ ID NO: 15) with a K_{d,app} of 192±20 nM, while no binding was observed to a base-paired RNA that does not contain internal loops (K_{d}>20 μM) (FIG. 3D & 3E).

Because 63 selectively binds to r(CCUG)₁₂ (SEQ ID NO: 15) over a base-paired control, its ability to improve other DM2-associated defects was assessed in DM2 patient-derived fibroblasts. Compound 63 (5 mM) reduced the number of r(CCUG)^{exp}-MBNL1 nuclear foci per cell from 7±1 in untreated patient-derived fibroblasts to 5±0.5 in treated cells, or an ˜30% reduction, as determined by RNA fluorescence in situ hybridization (FISH) and MBNL1 immunofluorescence (FIGS. 4A and B). This decrease in the number of nuclear foci correlates with the improvement in IR receptor splicing observed at 5 μM (FIG. 3D) as MBNL1 is no longer sequestered in foci and can resume its normal function. Importantly, 63 did not affect MAP4K4 exon 22a splicing, a NOVA-dependent splicing event (Figure S13).²⁵

As mentioned above, r(CCUG)^{exp} causes the aberrant retention of the intron in which it is harbored in the mature mRNA. We have previously shown that small molecules that bind r(CCUG)^{exp} can rescue defects in intron retention by facilitating removal of the retained intron, which is subsequently degraded.¹⁰ Thus, we evaluated 63's ability to rescue r(CCUG)^{exp}-mediated intron retention. Indeed, 63 significantly reduced the abundance of CNBP intron 1 in DM2 patient-derived fibroblasts at doses of 5 and 1 M (FIG. 4C), as determined by RT-qPCR. Importantly, 63 did not affect the levels of CNBP mature mRNA (FIG. 4D). The effects on intron 1 were also specific to mutant CNBP containing r(CCUG)^{exp}, as no effect on intron 1 was observed in wild-type fibroblasts (Figure S14). Furthermore, 63 did not affect IR exon 11 inclusion in wild-type fibroblasts (Figure S14). Thus, 63 specifically improved r(CCUG)^{exp}-mediated defects that are dysregulated in DM2 including nuclear foci, IR splicing, and intron retention.

Implications for Targeted Degradation of r(CCUG)^{exp}.

Importantly, all of the bioactive compounds identified herein display drug-like properties. The lead optimized compound, 63, has a low molecular weight (379.48 g/mol) and has a similar number of hydrogen bond donors (3) to known drugs in DrugBank (5). Furthermore, 63 has a favorable cLogP (3.75) and TPSA (83 Å) and satisfies the Rule of 5 criteria for drug-likeness. Through medicinal chemistry optimization of screening hits, we identified compounds with >10-fold improvement in in vitro activity for disruption of an r(CCUG)₁₂-MBNL1 complex. The most bioactive compound from lead optimization displayed ˜5-fold improvement in its ability to improve DM2-associated splicing defects. Importantly, this lead optimized compound (63) also rescued r(CCUG)^{exp}-containing intron 1 retention, resulting in degradation of the expanded repeat but not the mature transcript.

Targeted degradation is an emerging field for affecting the biological function of disease-causing RNAs. Thus far, targeted RNA degradation can be achieved through: (i) direct cleavage of RNAs through conjugation of an RNA-binding module to bleomycin or derivates of bleomycin;^{13, 26} (ii) recruitment of a ribonuclease through ribonuclease targeting chimeras (RIBOTACs), comprising an RNA-binding module and a compound that recruits RNaseL;^{14, 27} and (iii) small molecules that degrade repeat-containing introns, likely by increasing the accessibility of the disease-causing RNA to endogenous decay mechanisms. 10, 12 As the former two approaches require chimeric compounds, the molecular weight of such modalities are larger and can negatively alter physiochemical properties. Targeted degradation through endogenous RNA decay mechanisms, however, can be accomplished with monomeric small molecules.

According to the invention, it is possible to degrade r(CCUG)^{exp} with drug-like quinazoline core molecules. Targeted degradation of RNA repeat expansions with monomeric compounds can have broad applications as other repeat expansions, such as the r(G₄C₂)^{exp} that causes ALS/FTD or r(CUG)^{exp} that causes FECD, which are both found in retained introns.^{4, 28} Furthermore, targeted degradation of RNA through RNA quality control mechanisms may be broadly applicable with monomeric, drug-like small molecules that recognize RNA structures.

### Mechanism of Action and Medical Treatment

In certain embodiments, the invention is directed to methods of inhibiting, suppressing, depressing and/or managing aberrant r(CCUG)^{exp} by disrupting an r(CCUG)₁₂-MBNL1 complex and facilitating the repeat expansion's subsequent degradation via endogenous decay mechanisms. Because r(CCUG)^{exp} causes aberrant retention of the intron in which it is harbored in the pre-mRNA, thus misdirecting pre-mRNA splicing, the embodiments of the invention can rescue these defects by facilitating removal of the retained aberrant fragments such as an intron. In particular, compound 63 has been shown to have significant rescue ability, has low or no toxicity at effective dosage level and is highly specific for the target segment r(CCUG)^{exp}. A disease responding to this treatment is myotonic dystrophy type 2.

Embodiments of the quinazoline compounds of Formula I applied in methods of the invention and their pharmaceutical compositions are capable of acting as “arrestors” of r(CCUG)^{exp} which means that they are capable of reducing the severity of medical maladies caused by the defect.

The compounds useful for methods of the invention and their pharmaceutical compositions function as therapeutic agents in that they are capable of preventing, ameliorating, modifying and/or affecting a disorder or condition. The characterization of such compounds as therapeutic agents means that, in a statistical sample, the compounds reduce the occurrence of the disorder or condition in the treated sample relative to an untreated control sample or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as an DM2 disease may be accomplished according to the embodiments of the methods of the invention and includes administration of a composition as described above which reduces, or delays or inhibits or retards the deleterious medical condition in an DM2 subject relative to a subject which does not receive the composition.

The compounds of the present invention and their salts and solvates, thereof, may be employed alone or in combination with other therapeutic agents for the treatment of the diseases or conditions associated with r(CCUG)^{exp} sequestering of pre-RNA splicing activator/suppressor protein such as MBNL1.

The compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, subcutaneous or intrathecal), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating Compounds of Formula I

The pharmaceutical compositions of the invention incorporate embodiments of quinazoline compounds of Formula I, preferably the especially preferred embodiment of Formula I, useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these quinazoline compounds of Formula I depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
   (3) humectants, such as glycerol;
   (4) disintegrating agents, such as agar-agar, calcium carbonate, potato or tapioca starch, alginic acid, certain silicates, and sodium carbonate;
   (5) solution retarding agents, such as paraffin;
   (6) absorption accelerators, such as quaternary ammonium compounds;
   (7) wetting agents, such as, for example, acetyl alcohol and glycerol monostearate; (8) absorbents, such as kaolin and bentonite clay;
   (9) lubricants, such a talc, calcium stearate, magnesium stearate, solid polyethylene glycols, sodium lauryl sulfate, and mixtures thereof; and
   (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically.” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### EXPERIMENTAL EXAMPLES

### Materials and Methods

Abbreviations: CNBP, CCHC-Type Zinc Finger Nucleic Acid Binding Protein; DCM, dichloromethane; DIPEA, N,N-diisopropylethylamine; DMF, dimethylformamide; DM2, myotonic dystrophy type 2; HCl, hydrochloric acid; ESI, electrospray ionization; EtOH, ethanol; FISH, fluorescence in situ hybridization; HPLC, high performance liquid chromatography; IPA, isopropanol; IR, insulin receptor; LCMS, liquid chromatography mass spectrometry; MBNL1, muscleblind like 1; MeOH, methanol; MW, microwave; NMR, nuclear magnetic resonance; TFA, trifluoroacetic acid; TR-FRET, time-resolved fluorescence resonance energy transfer.

General synthetic procedures: Reagents and solvents were purchase from commercial sources and used without further purification. Microwave (MW)-assisted reactions were performed by an Initiator+(Biotage).

Compounds were purified: (i) by Isolera One flash chromatography system (Biotage) using pre-packed C18 columns (spherical 20-35 μm, Agela Technologies) or pre-packed silica gel columns (spherical 20-35 μm, Agela Technologies) or (ii) by HPLC (Waters 2489 and 1525) using a SunFire® Prep C18 OBD™ 5 μm column (19×150 mm) with a 5 mL/min flow. HPLC purity analysis was performed using a SunFire® C18 3.5 μm column (4.6×150 mm) with a linear gradient (0%-100% methanol (MeOH)+0.1% (v/v) trifluoracetic acid (TFA) and water +0.1% (v/v) TFA over 60 min at flow rate of 1 mL/min.

NMR spectra were collected on a 400 UltraShield™ (Bruker) (400 MHz for 1H and 100 MHz for ¹³C) or Ascend™ 600 (Bruker) (600 MHz for 1H and 150 MHz for ¹³C). Chemical shifts are reported in ppm relative to tetramethylsilane (TMS) for 1H and residual solvent for ¹³C as internal standards. Coupling constant (J values) are expressed in Hz. High resolution mass spectra were recorded on an Agilent 1260 Infinity LC system coupled to an Agilent 6230 TOF (HR-ESI) with a Poroshell 120 EC-C18 column (Agilent, 50 mm×4.6 mm, 2.7 μm). LCMS analysis was performed by Agilent 1260 Infinity LC system coupled to an Agilent 6130 quadrupole LC/MS (ESI) with a ZORBAX SB—C18 column (Agilent, 50 mm×2.1 mm, 1.8 μm). Compounds 1-44 were purchased from ChemBridge and used without further purification.

### Synthetic Schemes

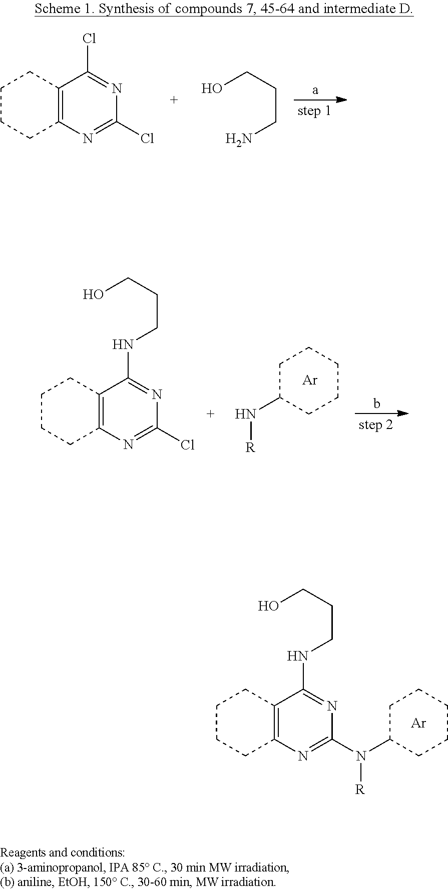

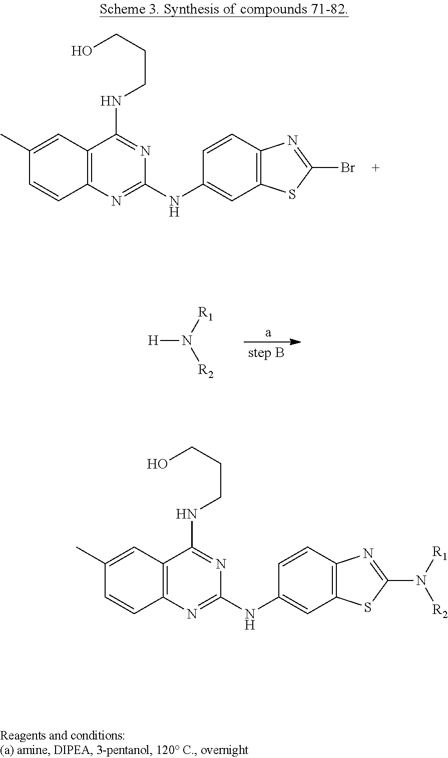

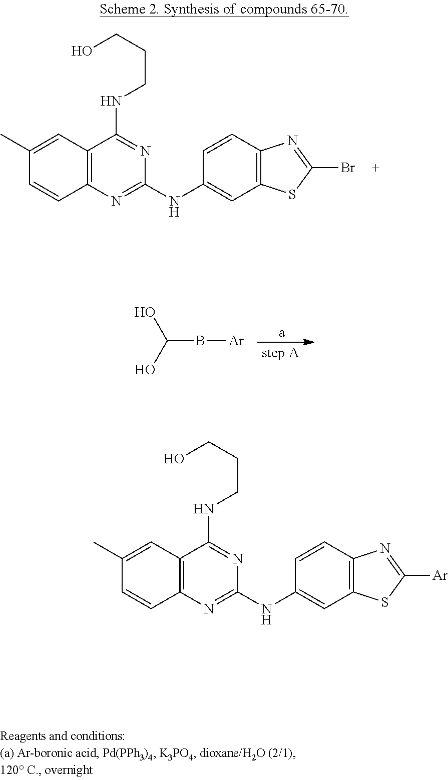

General synthetic procedure for 7, 45-64: For step 1, a mixture of dichloroquinazoline (A) (0.469 mmol, 1.0 equiv) and 3-aminopropan-1-ol (1.52 mmol, 3.2 equiv) in IPA (0.40 M) was heated at 85° C. for 30 min under MW irradiation. The reaction was cooled to room temperature, and product formation was confirmed by LCMS. The reaction mixture was then concentrated in vacuo and washed with H₂O (4 mL×2) and dried to afford an intermediate B. The material was used in the next reaction without further purification. Then in step 2, a mixture of B (79.5 μmol, 1.0 equiv) and a corresponding aniline (C) (159 μmol, 2.0 equiv) in ethanol (EtOH) was heated at 150° C. for 30-60 min under MW irradiation. The reaction was cooled to room temperature, and product formation was confirmed by LCMS. The reaction mixture was then concentrated in vacuo and purified by column chromatography [Agela Technologies, Silica, 20 g, 0%-30% MeOH in dichloromethane (DCM)] to afford compounds 45-64.

Synthesis of 7: Following general synthetic procedure 1 using 2,4-dichloroquinazoline (93.3 mg, 0.469 mmol) in step 1 (obtained monochloro intermediate; 105.0 mg, 94%) and 2,3-dihydrobenzo[b][1,4]dioxin-6-amine (24.0 mg, 159 μmol) in step 2 (18.9 mg, 79.5 μmol of monochloro intermediate) afforded 3-((2-((2,3-dihydrobenzo[b][1,4]dioxin-6-yl)amino)quinazolin-4-yl)amino)propan-1-ol (7) (12.2 mg, 44%) ¹H NMR (400 MHZ, MeOD) δ 8.08 (d, J=8.2 Hz, 1H), 7.78 (d, J=7.4 Hz, 1H), 7.52 (d, J=8.2 Hz, 1H), 7.45 (d, J=7.4 Hz, 1H), 7.11-6.86 (m, 3H), 4.28 (s, 4H), 3.76 (t, J=7.1 Hz, 2H), 3.67 (t, J=6.1 Hz, 2H), 2.00-1.90 (m, 2H) ¹³C NMR (150 MHz, DMSO-d₆) δ 160.4, 154.7, 145.4, 143.5, 140.0, 134.2, 133.1, 123.9, 123.3, 121.7, 117.2, 114.4, 111.4, 110.1, 64.7, 64.4, 59.0, 38.9, 32.1; HR-MS (ESI): Calcd for C₁₉H₁₉N₄O₃⁻ [M−H]⁻; 351.1463; found, 351.1463.

Synthesis of 45: Following general synthetic procedure 1 using (93.3 mg, 0.469 mmol) in step 1 (obtained monochloro intermediate; 105.0 mg. 94%) and benzo[d][1.3]dioxol-5-amine (21.8 mg. 159 μmol) in step 2 (18.9 mg, 79.5 μmol of monochloro intermediate) afforded 3-((2-((4-methyl-3,4-dihydro-2H-benzo[b][1,4]oxazin-7-yl)amino)quinazolin-4-yl)amino)propan-1-ol (45) (16.6 mg, 62%) ¹H NMR (400 MHZ, DMSO-d₆) δ 10.4 (br s, 1H), 9.89 (br s, 1H), 8.48-8.37 (m, 1H), 7.83-7.73 (m, 1H), 7.55-7.47 (m, 1H), 7.45-7.29 (m, 2H), 7.05-6.97 (m, 1H), 6.97-6.91 (m, 1H), 6.06 (s, 2H), 4.75-4.57 (m, 1H), 3.65-3.59 (m, 2H), 3.53-3.47 (m, 2H), 1.87-1.78 (m, 2H); HR-MS (ESI): Calcd for C18H₁₇N₄O₃⁻ [M−H]⁻; 337.1306; found, 337.1321.

Synthesis of 46: Following general synthetic procedure 1 using 2,4-dichloroquinazoline (93.3 mg. 0.469 mmol) in step 1 (obtained monochloro intermediate; 105.0 mg. 94%) and 2,2-difluorobenzo[d][1,3]dioxol-5-amine (27.5 mg, 159 μmol) in step 2 (18.9 mg, 79.5 μmol of monochloro intermediate) afforded 3-((2-((2,2-difluorobenzo[d][1,3]dioxol-5-yl)amino)quinazolin-4-yl)amino)propan-1-ol (46) (12.4 mg, 42%) ¹H NMR (400 MHZ, DMSO-d₆) δ 10.5 (br s, 1H), 9.77 (br s, 1H), 8.40-8.33 (m, 1H), 7.91-7.75 (m, 2H), 7.60-7.56 (m, 1H), 7.51-7.41 (m, 2H), 7.37-7.31 (m, 1H), 4.79-4.43 (m, 1H), 3.69-3.58 (m, 2H), 3.57-3.46 (m, 2H), 1.87-1.78 (m, 2H); HR-MS (ESI): Calcd for C₁₈H₁₅F₂N₄O₃⁻ [M−H]⁻; 373.1118; found, 373.1119.

Synthesis of 47: Following general synthetic procedure 1 using 2,4-dichloroquinazoline (93.3 mg, 0.469 mmol) in step 1 (obtained monochloro intermediate; 105.0 mg, 94%) and 5-methyl-2,3-dihydrobenzo[b][1.4]dioxin-6-amine (26.3 mg, 159 μmol) in step 2 (18.9 mg. 79.5 μmol of monochloro intermediate) afforded 3-((2-((5-methyl-2,3-dihydrobenzo[b][1.4]dioxin-6-yl)amino)quinazolin-4-yl)amino)propan-1-ol (47) (29.0 mg, 100%) ¹H NMR (400 MHZ, DMSO-d₆) δ 9.86-8.69 (m, 2H), 8.24 (d, J=7.8 Hz, 1H), 7.72-7.65 (m, 1H), 7.46 (d. J=7.6 Hz, 1H), 7.35-7.27 (m, 1H), 6.96 (d. J=8.6 Hz, 1H), 6.75 (d. J=8.6 Hz. 1H), 4.58-4.54 (m, 1H), 4.33-4.28 (m, 2H), 4.27-4.22 (m, 2H), 3.59-3.49 (m, 2H), 3.49-3.43 (m, 2H), 2.06 (s, 3H), 1.80-1.73 (m, 2H); HR-MS (ESI): Calcd for C₂₀H₂₁N₄O₃⁻ [M−H]⁻; 365.1619; found, 365.1622.

Synthesis of 48: Following general synthetic procedure 1 using 2,4-dichloroquinazoline (93.3 mg. 0.469 mmol) in step 1 (obtained monochloro intermediate; 105.0 mg, 94%) and 4-methyl-3,4-dihydro-2H-benzo[b][1,4]oxazin-7-amine (26.1 mg, 159 μmol) in step 2 (18.9 mg. 79.5 μmol of monochloro intermediate) afforded 3-((2-((4-methyl-3,4-dihydro-2H-benzo[b][1,4]oxazin-7-yl)amino)quinazolin-4-yl)amino)propan-1-ol (48) (6.3 mg, 22%) ¹H NMR (400 MHZ, MeOD) δ 7.86-7.82 (m, 1H), 7.86-7.82 (m, 1H), 7.57-7.52 (m, 1H), 7.39-7.35 (m, 1H), 7.20 (d. J=2.4 Hz, 1H), 7.16-7.11 (m, 1H), 7.01 (dd, J=8.6, 2.5 Hz, 1H), 6.70 (d. J=8.7 Hz, 1H), 4.31-4.27 (m, 1H), 3.72-3.65 (m, 4H), 3.21-3.17 (m, 2H), 2.84 (s, 3H), 1.97-1.88 (m, 2H); HR-MS (ESI): Calcd for C₂₀H₂₂N₅O₂⁻ [M−H]⁻; 364.1779; found, 364.1786.

Synthesis of 49: Following general synthetic procedure 1 using 2,4-dichloroquinazoline (93.3 mg, 0.469 mmol) in step 1 (obtained monochloro intermediate; 105.0 mg, 94%) and 2,3-dihydrobenzo[b][1,4]dioxin-6-amine (23.5 mg, 159 μmol) in step 2 (18.9 mg, 79.5 μmol of monochloro intermediate) afforded 3-((2-((2-methylbenzo[d]oxazol-6-yl)amino)quinazolin-4-yl)amino)propan-1-ol (49) (8.2 mg, 30%) ¹H NMR (400 MHZ, MeOD) δ 8.29 (d, J=1.8 Hz, 1H), 7.94 (dd, J=8.2, 1.0 Hz, 1H), 7.68-7.62 (m, 1H), 7.54-7.45 (m, 2H), 7.43 (dd, J=8.6, 2.0 Hz, 1H), 7.29-7.23 (m, 1H), 3.75 (t, J=7.0 Hz, 2H), 3.69 (t, J=6.2 Hz, 2H), 2.63 (s, 3H), 2.00-1.92 (m, 2H) ¹³C NMR (150 MHz, DMSO-d₆) δ 163.1, 160.6, 151.2, 138.6, 135.8, 133.6, 128.5, 126.0, 124.3, 123.5, 122.7, 118.8, 117.0, 112.0, 101.6, 59.1, 38.7, 32.3, 14.6; HR-MS (ESI): Calcd for C₁₉H₁₈N₅O₂⁻ [M−H]⁻; 348.1466; found, 348.1470.

Synthesis of 50: Following general synthetic procedure 1 using 2,4-dichloroquinazoline (93.3 mg, 0.469 mmol) in step 1 (obtained monochloro intermediate; 105.0 mg, 94%) and 2-methylbenzo[d]thiazol-6-amine (26.1 mg. 159 μmol) in step 2 (18.9 mg, 79.5 μmol of monochloro intermediate) afforded 3-((2-((2-methylbenzo[d]thiazol-6-yl)amino)quinazolin-4-yl)amino)propan-1-ol (50) (7.7 mg, 27%) ¹H NMR (400 MHZ, MeOD) δ 8.56 (d, J=2.0 Hz, 1H), 7.93-7.88 (m, 1H), 7.70 (d, J=8.8 Hz, 1H), 7.66-7.63 (m, 1H), 7.63-7.57 (m, 1H), 7.49-7.45 (m, 1H), 7.24-7.17 (m, 1H), 3.74 (t, J=7.0 Hz, 2H), 3.70 (t. J=6.2 Hz, 2H), 2.79 (s, 3H), 2.00-1.92 (m, 2H) ¹³C NMR (150 MHz, DMSO-d₆) δ 163.8, 160.6, 157.4, 151.4, 147.6, 139.5, 136.3, 133.0, 125.8, 123.2, 122.0, 121.9, 118.7, 112.2, 110.1, 59.2, 38.5, 32.4, 20.1; HR-MS (ESI): Calcd for C₁₉H₁₈N₅OS⁻ [M−H]⁻; 364.1238; found, 364.1245.

Synthesis of 51: Following general synthetic procedure 1 using 2,4-dichloroquinazoline (93.3 mg, 0.469 mmol) in step 1 (obtained monochloro intermediate; 105.0 mg. 94%) and 1-methyl-1H-indazol-6-amine (23.4 mg. 159 μmol) in step 2 (18.9 mg, 79.5 μmol of monochloro intermediate) afforded 3-((2-((1-methyl-1H-indazol-6-yl)amino)quinazolin-4-yl)amino)propan-1-ol (51) (25.4 mg, 92%) ¹H NMR (400 MHZ, MeOD) δ 8.31 (br s, 1H), 7.97-7.93 (m, 1H), 7.89 (d, J=0.9 Hz, 1H), 7.67-7.61 (m, 2H), 7.52-7.48 (m, 1H), 7.28-7.21 (m, 2H), 3.10 (s, 3H), 3.80 (t, J=6.9 Hz, 2H), 3.69 (t, J=6.1 Hz, 2H), 2.02-1.94 (m, 2H); HR-MS (ESI): Calcd for C₁₉H₁₉N₆O⁻ [M−H]⁻; 347.1626; found, 347.1630.

Synthesis of 52: Following general synthetic procedure 1 using 2,4-dichloroquinazoline (93.3 mg. 0.469 mmol) in step 1 (obtained monochloro intermediate; 105.0 mg, 94%) and 1-methyl-1H-indazol-3-amine (23.4 mg. 159 μmol) in step 2 (18.9 mg, 79.5 μmol of monochloro intermediate) afforded 3-((2-((1-methyl-1H-indazol-3-yl)amino)quinazolin-4-yl)amino)propan-1-ol (52) (5.2 mg. 19%) ¹H NMR (400 MHz, DMSO-d₆) δ 12.8 (br s, 0.5H), 11.5 (br s, 0.5H), 9.87 (br s, 1H), 8.51 (d, J=8.1 Hz, 1H), 8.02 (d, J=8.1 Hz, 1H), 7.90-7.82 (m, 1H), 7.79-7.72 (m, 1H), 7.68 (d, J=8.6 Hz, 1H), 7.55-7.45 (m, 2H), 7.23-7.15 (m, 1H), 4.72-4.56 (m, 1H), 4.10 (3H, s), 3.75-3.56 (m, 2H), 3.52-3.39 (m, 2H), 1.88-1.70 (m, 2H); HR-MS (ESI): Calcd for C₁₉H₁₉N₆O⁻ [M−H]⁻; 347.1626; found, 347.1635.

Synthesis of 53: Following general synthetic procedure 1 using 2,4-dichloroquinazoline (93.3 mg, 0.469 mmol) in step 1 (obtained monochloro intermediate; 105.0 mg, 94%) and N-methyl-2,3-dihydrobenzo[b][1,4]dioxin-6-amine (26.3 mg, 159 μmol) in step 2 (18.9 mg. 79.5 μmol of monochloro intermediate) afforded 3-((2-((2,3-dihydrobenzo[b][1,4]dioxin-6-yl)(methyl)amino)quinazolin-4-yl)amino)propan-1-ol (53) (7.5 mg. 26%) ¹H NMR (400 MHz, DMSO-d₆) δ 9.63 (br s, 1H), 8.36 (d, J=8.2 Hz, 1H), 7.75-7.69 (m, 2H), 7.40-7.34 (m, 1H), 7.04 (d, J=2.2 Hz, 1H), 7.00-6.91 (m, 2H), 4.65-4.58 (m, 1H), 4.32-4.28 (m, 4H), 3.58-3.49 (m, 2H), 3.52 (s, 3H), 3.48-3.41 (m, 2H), 1.81-1.70 (m, 2H); HR-MS (ESI): Calcd for C₂₀H₂₁N₄O₃⁻ [M−H]⁻; 365.1619; found, 365.1636.

Synthesis of 54: Following general synthetic procedure 1 using 2,4-dichloro-5,6,7,8-tetrahydroquinazoline (95.2 mg, 0.469 mmol) in step 1 (obtained monochloro intermediate; 86.3 mg. 76%) and 2,3-dihydrobenzo[b][1,4]dioxin-6-amine (24.0 mg, 159 μmol) in step 2 (19.2 mg. 79.5 μmol of monochloro intermediate) afforded 3-((2-((2,3-dihydrobenzo[b][1.4]dioxin-6-yl)amino)-5,6,7,8-tetrahydroquinazolin-4-yl)amino)propan-1-ol (54) (15.4 mg, 54%) ¹H NMR (400 MHZ, DMSO-d₆) δ 9.40 (br s, 1H), 7.60 (br s, 1H), 7.30 (d, J=2.2 Hz, 1H), 7.01 (dd, J=8.8, 2.2 Hz, 1H), 6.77 (d, J=8.8 Hz, 1H), 4.54 (t, J=4.7 Hz, 1H), 4.26-4.17 (m, 4H), 3.51-3.42 (m, 4H), 2.54-2.46 (m, 2H), 2.28-2.21 (m, 2H), 1.79-1.66 (m, 6H); HR-MS (ESI): Calcd for C₁₉H₂₃N₄O₃⁻ [M−H]⁻; 355.1778; found, 355.1773.

Synthesis of 55: Following general synthetic procedure 1 using 2,4-dichloro-5,7-dihydrofuro[3,4-d]pyrimidine (89.6 mg. 0.469 mmol) in step 1 (obtained monochloro intermediate; 88.8 mg. 82%) and 2,3-dihydrobenzo[b][1,4]dioxin-6-amine (24.0 mg, 159 μmol) in step 2 (18.2 mg, 79.5 μmol of monochloro intermediate) afforded 3-((2-((2,3-dihydrobenzo[b][1,4]dioxin-6-yl)amino)-5,7-dihydrofuro[3,4-d]pyrimidin-4-yl)amino)propan-1-ol (55) (7.8 mg. 29%) ¹H NMR (400 MHZ, DMSO-d₆) δ 8.86 (br s, 1H), 7.45 (d, J=2.5 Hz, 1H), 7.12 (dd, J=8.8, 2.5 Hz, 1H), 7.02 (t, J=5.2 Hz, 1H), 6.69 (d, J=8.8 Hz, 1H), 4.80 (br s, 2H), 4.68-4.63 (m, 2H), 4.45 (t. J=4.6 Hz, 1H), 4.23-4.13 (m, 4H), 3.53-3.38 (m, 4H), 1.77-1.68 (m. 2H); HR-MS (ESI): Calcd for C₁₇H₁₉N₄O₄⁻ [M−H]⁻; 343.1412; found, 343.1418.

Synthesis of 56: Following general synthetic procedure 1 using 4,6-dichloro-3-methyl-1H-pyrazolo[3,4-d]pyrimidine (95.2 mg, 0.469 mmol) in step 1 (obtained monochloro intermediate; 44.0 mg. 39%) and 2,3-dihydrobenzo[b][1,4]dioxin-6-amine (24.0 mg, 159 μmol) in step 2 (19.2 mg. 79.5 μmol of monochloro intermediate) afforded 3-((6-((2,3-dihydrobenzo[b][1,4]dioxin-6-yl)amino)-3-methyl-1H-pyrazolo[3,4-d]pyrimidin-4-yl)amino)propan-1-ol (56) (12.5 mg, 44%) ¹H NMR (400 MHZ, DMSO-d₆) δ 12.4 (br s, 1H), 8.79 (br s, 1H), 7.62-7.41 (m, 1H), 7.20-7.05 (m, 1H), 6.91 (br s, 1H), 6.79-6.65 (m, 1H), 4.84-4.49 (m, 1H), 4.28-4.13 (m, 4H), 3.64-3.50 (m, 4H), 2.45 (br s, 3H), 1.85-1.74 (m, 2H); HR-MS (ESI): Calcd for C₁₇H₁₉N₆O₃⁻ [M−H]⁻; 355.1524; found, 355.1537.

Synthesis of 57: Following general synthetic procedure 1 using 2,4-dichloro-8-methylquinazoline (100.0 mg. 0.469 mmol) in step 1 (obtained monochloro intermediate; 104.6 mg, 89%) and 2,3-dihydrobenzo[b][1,4]dioxin-6-amine (24.0 mg. 159 μmol) in step 2 (20.0 mg, 79.5 μmol of monochloro intermediate) afforded 3-((2-((2,3-dihydrobenzo[b][1,4]dioxin-6-yl)amino)-8-methylquinazolin-4-yl)amino)propan-1-ol (57) (4.2 mg. 14%) ¹H NMR (400 MHZ, MeOD) δ 7.95 (d. J=7.9 Hz, 1H), 7.34 (d. J=7.3 Hz, 1H), 7.39-7.35 (m, 1H), 7.29-7.24 (m, 1H), 6.99 (d, J=8.7, 2.6 Hz, 1H), 6.89 (d. J=8.7 Hz, 1H), 4.31-4.25 (m, 4H), 3.78-3.74 (m, 2H), 3.67 (t, J=6.2 Hz, 2H), 2.51 (s, 3H), 1.99-1.91 (m, 2H); HR-MS (ESI): Calcd for C₂₀H₂₁N₄O₃⁻ [M−H]⁻; 365.1619; found, 365.1628.

Synthesis of 58: Following general synthetic procedure 1 using 2,4-dichloro-6-methylquinazoline (100.0 mg, 0.469 mmol) in step 1 (obtained monochloro intermediate; 103.8 mg. 88%) and 2,3-dihydrobenzo[b][1,4]dioxin-6-amine (24.0 mg. 159 μmol) in step 2 (20.0 mg, 79.5 μmol of monochloro intermediate) afforded 3-((2-((2,3-dihydrobenzo[b][1,4]dioxin-6-yl)amino)-6-methylquinazolin-4-yl)amino)propan-1-ol (58) (9.0 mg. 31%) ¹H NMR (400 MHZ, MeOD) δ 7.90 (br s, 1H), 7.65-7.60 (m, 1H), 7.41 (d, J=8.5 Hz, 1H), 7.06 (br s, 1H), 6.95-6.88 (m, 2H), 4.27 (s, 4H), 3.75 (t. J=7.1 Hz, 2H), 3.67 (t. J=6.2 Hz, 2H), 2.47 (s, 3H), 1.99-1.89 (m, 2H) ¹³C NMR (150 MHz, DMSO-d₆) δ 160.2, 152.4, 143.7, 140.9, 138.7, 136.4, 134.1, 131.3, 123.8, 118.6, 117.5, 115.6, 111.3, 110.7, 64.7, 64.5, 58.8, 39.3, 31.9, 21.2; HR-MS (ESI): Calcd for C₂₀H₂₁N₄O₃⁻ [M−H]⁻; 365.1619; found, 365.1622.

Synthesis of 59: Following general synthetic procedure 1 using 2,4-dichloro-6-fluoroquinazoline (101.8 mg, 0.469 mmol) in step 1 (obtained monochloro intermediate; 93.8 mg, 78%) and 2,3-dihydrobenzo[b][1,4]dioxin-6-amine (24.0 mg, 159 μmol) in step 2 (20.3 mg, 79.5 μmol of monochloro intermediate) afforded 3-((2-((2,3-dihydrobenzo[b][1,4]dioxin-6-yl)amino)-6-fluoroquinazolin-4-yl)amino)propan-1-ol (59) (8.0 mg, 27%) ¹H NMR (400 MHZ, DMSO-d₆) δ 9.59 (br s, 1H), 8.92 (br s, 1H), 8.19-8.08 (m, 1H), 7.65-7.56 (m, 1H), 7.56-7.48 (m. 1H), 7.42-7.28 (m, 1H), 7.16-7.05 (m, 1H), 6.82 (d, J=8.7 Hz, 1H), 4.64-4.50 (m, 1H), 4.29-4.18 (m, 4H), 3.65-3.56 (m, 2H), 3.56-3.49 (m, 2H), 1.87-1.78 (m, 2H); HR-MS (ESI): Calcd for C₁₉H₁₈FN₄O₃⁻[M−H]⁻; 369.1368; found, 369.1375.

Synthesis of 60: Following general synthetic procedure 1 using 2,4-dichloro-7-fluoroquinazoline (101.8 mg, 0.469 mmol) in step 1 (obtained monochloro intermediate; 46.1 mg, 38%) and 2,3-dihydrobenzo[b][1,4]dioxin-6-amine (24.0 mg, 159 μmol) in step 2 (20.3 mg, 79.5 μmol of monochloro intermediate) afforded 3-((2-((2,3-dihydrobenzo[b][1,4]dioxin-6-yl)amino)-7-fluoroquinazolin-4-yl)amino)propan-1-ol (60) (3.0 mg. 10%) ¹H NMR (400 MHZ, MeOD) δ 8.21-8.12 (m, 1H), 7.31-7.19 (m, 2H), 7.11-7.00 (m, 1H), 6.98-6.87 (m, 2H), 4.28 (s, 4H), 3.75 (t, J=7.1 Hz, 2H), 3.67 (t, J=6.1 Hz, 2H), 1.99-1.90 (m, 2H); HR-MS (ESI): Calcd for C₁₉H₁₈FN₄O₃⁻ [M−H]⁻; 369.1368; found, 369.1383.

Synthesis of 61: Following general synthetic procedure 1 using 2,4-dichloro-8-fluoroquinazoline (101.8 mg. 0.469 mmol) in step 1 (obtained monochloro intermediate; 76.5 mg, 64%) and 2,3-dihydrobenzo[b][1,4]dioxin-6-amine (24.0 mg. 159 μmol) in step 2 (20.3 mg. 79.5 μmol of monochloro intermediate was used) afforded 3-((2-((2,3-dihydrobenzo[b][1,4]dioxin-6-yl)amino)-8-fluoroquinazolin-4-yl)amino)propan-1-ol (61) (11.5 mg, 39%) ¹H NMR (400 MHZ, DMSO-d₆) δ 9.21 (br s, 1H), 8.38 (br s, 1H), 7.91 (d, J=8.2 Hz, 1H), 7.73-7.56 (m, 1H), 7.51-7.40 (m, 1H), 7.30-7.21 (m, 1H), 7.17-7.07 (m, 1H), 6.75 (d, J=8.7 Hz, 1H), 4.64-4.47 (m, 1H), 4.30-4.15 (m, 4H), 3.66-3.48 (m, 4H), 1.90-1.78 (m, 2H); HR-MS (ESI): Calcd for C₁₉H₁₈FN₄O₃⁻ [M−H]⁻; 369.1368; found, 369.1383.

Synthesis of 62: Following general synthetic procedure 1 using 2,4-dichloro-6-methylquinazoline (100.0 mg, 0.469 mmol) in step 1 (obtained monochloro intermediate; 103.8 mg. 88%) and 2-methylbenzo[d]oxazol-6-amine (23.5 mg. 159 μmol) in step 2 (20.0 mg, 79.5 μmol of monochloro intermediate) afforded 3-((6-methyl-2-((2-methylbenzo[d]oxazol-6-yl)amino)quinazolin-4-yl)amino)propan-1-ol (62) (6.2 mg, 21%) ¹H NMR (400 MHZ, MeOD) δ 8.11 (d, J=1.8 Hz, 1H), 7.84 (br s, 1H), 7.60-7.55 (m, 2H), 7.44-7.38 (m, 2H), 3.75 (t. J=7.0 Hz, 2H), 3.67 (t, J=6.1 Hz, 2H), 2.64 (s, 3H), 2.46 (s, 3H), 2.00-1.92 (m, 2H) ¹³C NMR (150 MHz, DMSO-d₆) δ 163.8, 160.3, 154.1, 151.1, 136.9, 135.8, 133.2, 128.6, 126.0, 123.3, 121.5, 119.1, 117.9, 111.3, 102.9, 58.9, 39.0, 32.0, 21.3, 14.6; HR-MS (ESI): Calcd for C₂₀H₂₀N₅O₂⁻ [M−H]⁻; 362.1623; found, 362.1624.

Synthesis of 63: Following general synthetic procedure 1 using 2,4-dichloro-6-methylquinazoline (100.0 mg, 0.469 mmol) in step 1 (obtained monochloro intermediate; 103.8 mg 88%) and 2-methylbenzo[d]thiazol-6-amine (26.1 mg, 159 μmol) in step 2 (20.0 mg. 79.5 μmol of monochloro intermediate) afforded 3-((6-methyl-2-((2-methylbenzo[d]thiazol-6-yl)amino)quinazolin-4-yl)amino)propan-1-ol (63) (15.4 mg. 56%) ¹H NMR (400 MHZ, MeOD) δ 8.31 (d. J=2.0 Hz, 1H), 7.91-7.85 (m, 2H), 7.62-7.58 (m, 2H), 7.41 (d. J=8.5 Hz, 1H), 3.76 (t, J=7.1 Hz, 2H), 3.66 (t, J=6.1 Hz, 2H), 2.83 (s, 3H), 2.47 (s, 3H), 1.98-1.90 (m, 2H) ¹³C NMR (150 MHz, DMSO-d₆) δ 164.7, 160.3, 155.4, 148.5, 145.9, 138.0, 136.2, 135.2, 132.2, 123.5, 122.8, 122.0, 119.6, 111.6, 59.1, 38.8, 32.3, 21.3, 20.1; HR-MS (ESI): Calcd for C₂₀H₂₂N₅OS⁻ [M−H]⁻; 378.1394; found, 378.1403.

Synthesis of 64: Following general synthetic procedure 1 using 2,4-dichloro-6-methylquinazoline (100.0 mg. 0.469 mmol) in step 1 (obtained monochloro intermediate; 103.8 mg, 88%) and 1-methyl-1H-indazol-6-amine (23.4 mg, 159 μmol) in step 2 (20.0 mg, 79.5 μmol of monochloro intermediate) afforded 3-((6-methyl-2-((1-methyl-1H-indazol-6-yl)amino)quinazolin-4-yl)amino)propan-1-ol (64) (7.9 mg, 27%) ¹H NMR (400 MHZ, MeOD) δ 8.27 (br s, 1H), 7.89 (d, J=0.9 Hz, 1H), 7.78 (br s, 1H), 7.67-7.63 (m, 1H), 7.53-7.48 (m, 1H), 7.41 (d, J=8.5 Hz, 1H), 7.24-7.20 (m, 1H), 4.04 (s, 3H), 3.79 (t, J=6.9 Hz, 2H), 3.69 (t, J=6.1 Hz, 2H), 2.45 (s, 3H), 2.02-1.93 (m, 2H); HR-MS (ESI): Calcd for C₂₀H₂₁N₆O⁻ [M−H]⁻; 361.1782; found, 361.1778.

Synthesis of intermediate D: Following general synthetic procedure 1 using 2,4-dichloro-6-methylquinazoline (1.50 g, 7.04 mmol) in step 1 (obtained monochloro intermediate; 1.56 g. 88%). and 2-bromobenzo[d]thiazol-6-amine (500 mg. 2.18 mmol) in step 2 (1.10 g, 4.37 mmol of monochloro intermediate) afforded 3-((2-((2-bromobenzo[d]thiazol-6-yl)amino)-6-methylquinazolin-4-yl)amino)propan-1-ol (D) (1.00 g, quant). ¹H NMR (400 MHZ, MeOD) δ 8.32-8.25 (m, 1H), 8.02-7.94 (m, 2H), 7.69 (dd, J=8.5, 1.5 Hz, 1H), 7.64 (dd, J=8.8, 2.2 Hz, 1H), 7.44 (d, J=8.5 Hz, 1H), 3.80-3.74 (m, 2H), 3.65 (t, J=6.1 Hz, 2H), 2.49 (s, 3H), 1.99-1.89 (m, 2H); HR-MS (ESI): Calcd for C₁₉H₁₈BrN₅OS⁺ [M+H]⁺; 444.0488; found, 444.04777.

General synthetic procedure for 65-70: A mixture of 3-((2-((2-bromobenzo[d]thiazol-6-yl)amino)-6-methylquinazolin-4-yl)amino)propan-1-ol (D) (20.0 mg, 45.0 μmol, 1.0 equiv), Ar-boronic acid (67.5 μmol, 1.5 equiv), Pd(PPh₃)₄ (5.2 mg, 4.5 μmol, 0.1 equiv) and K₃PO₄ (19.1 mg, 90.0 μmol, 2.0 equiv) in dioxane/H₂O (2/1, 0.50 mL) was heated at 120° C. overnight. The reaction was cooled to room temperature, and product formation was confirmed by LCMS. The reaction mixture was then concentrated in vacuo and purified by column chromatography as described above (Agela Technologies, Silica, 20 g, 0%-20% MeOH in DCM) and HPLC to afford compounds 65-70.

Synthesis of 65: Following general synthetic procedure 2 using D (20.0 mg, 45.0 μmol) and (4-fluorophenyl)boronic acid (9.4 mg, 67.5 μmol) afforded 3-((2-((2-(4-fluorophenyl)benzo[d]thiazol-6-yl)amino)-6-methylquinazolin-4-yl)amino)propan-1-ol (65) (5.0 mg. 24%). ¹H NMR (400 MHZ, DMSO-d₆) δ 12.6 (br s, 1H), 10.6 (br s, 1H), 9.55 (br s, 1H), 8.51 (br s, 1H), 8.19-8.06 (m, 4H), 7.73-7.65 (m, 2H), 7.54-7.40 (m, 3H), 4.79-4.49 (m, 1H), 3.72-3.63 (m, 2H), 3.57-3.49 (m, 2H), 2.43 (s, 3H), 1.90-1.80 (m, 2H); HR-MS (ESI): Calcd for C₂₅H₂₃FN₅OS⁺ [M+H]⁺; 460.1602; found, 460.1613.

Synthesis of 66: Following general synthetic procedure 2 using D (20.0 mg, 45.0 μmol) and (3-fluorophenyl)boronic acid (9.4 mg. 67.5 μmol) afforded 3-((2-((2-(3-fluorophenyl)benzo[d]thiazol-6-yl)amino)-6-methylquinazolin-4-yl)amino)propan-1-ol (66) (5.0 mg. 24%). ¹H NMR (400 MHZ, DMSO-d₆) δ 12.7 (br s, 1H), 10.6 (br s, 1H), 9.58 (br s, 1H), 8.57 (br s, 1H), 8.15-8.07 (m, 2H), 7.96-7.87 (m, 2H), 7.75-7.61 (m, 3H), 7.53-7.42 (m, 2H), 4.77-4.56 (m, 1H), 3.72-3.63 (m, 2H), 3.57-3.49 (m, 2H), 2.43 (s, 3H), 1.90-1.80 (m, 2H); HR-MS (ESI): Calcd for C₂₅H₂₃FN₅OS⁺ [M+H]⁺; 460.1602; found, 460.1621.

Synthesis of 67: Following general synthetic procedure 2 using D (20.0 mg, 45.0 μmol) and o-tolylboronic acid (9.2 mg, 67.5 μmol) afforded 3-((6-methyl-2-((2-(o-tolyl)benzo[d]thiazol-6-yl)amino)quinazolin-4-yl)amino)propan-1-ol (67) (9.4 mg, 46%). 1H NMR (400 MHZ, DMSO-d₆) δ 12.6 (br s, 1H), 10.6 (br s, 1H), 9.60 (br s, 1H), 8.53 (br s, 1H), 8.16-8.07 (m, 2H), 7.83 (d, J=7.3 Hz, 1H), 7.75-7.63 (m, 2H), 7.53-7.38 (m, 4H), 4.75-4.52 (m, 1H), 3.73-3.63 (m, 2H), 3.58-3.50 (m, 2H), 2.65 (s, 3H), 2.43 (s, 3H), 1.91-1.80 (m, 2H); HR-MS (ESI): Calcd for C₂₆H₂₆N₅OS⁺ [M+H]⁺; 456.1853; found, 456.1872.

Synthesis of 68: Following general synthetic procedure 2 using D (20.0 mg, 45.0 μmol) and (1H-pyrazol-3-yl)boronic acid (7.6 mg, 67.5 μmol) afforded 3-((2-((2-(1H-pyrazol-3-yl)benzo[d]thiazol-6-yl)amino)-6-methylquinazolin-4-yl)amino)propan-1-ol (68) (18.5 mg, 95%). ¹H NMR (400 MHZ, DMSO-d₆) δ 13.4 (s, 1H), 12.5 (br s, 1H), 10.6 (br s, 1H), 9.66 (br s, 1H), 8.42 (br s, 1H), 8.19-7.93 (m, 3H), 7.71-7.60 (m, 2H), 7.54-7.46 (m, 1H), 6.93 (s, 1H), 4.73-4.57 (m, 1H), 3.72-3.62 (m, 2H), 3.57-3.49 (m, 2H), 2.43 (s, 3H), 1.89-1.82 (m, 2H); HR-MS (ESI): Calcd for C₂₂H₂₂N₇OS⁺ [M+H]⁺; 432.1601; found, 432.1613.

Synthesis of 69: Following general synthetic procedure 2 using D (20.0 mg, 45.0 μmol) and (4-carbamoylphenyl)boronic acid (11.1 mg, 67.5 μmol) afforded 4-(6-((4-((3-hydroxypropyl)amino)-6-methylquinazolin-2-yl)amino)benzo[d]thiazol-2-yl)benzamide (69) (12.5 mg, 57%). ¹H NMR (400 MHZ, DMSO-d₆) δ 12.9 (br s, 1H), 10.7 (br s, 1H), 9.56 (br s, 1H), 8.59 (br s, 1H), 8.22-8.02 (m, 6H), 7.76-7.64 (m, 2H), 7.57 (s, 1H), 7.49 (d, J=8.4 Hz, 1H), 6.93 (s, 1H), 4.78-4.52 (m, 1H), 3.76-3.62 (m, 2H), 3.60-3.51 (m, 2H), 2.43 (s, 3H), 1.91-1.81 (m, 2H); HR-MS (ESI): Calcd for C₂₆H₂₅N₆O₂S⁺ [M+H]⁺; 485.1754; found, 485.1778.

Synthesis of 70: Following general synthetic procedure 2 using D (20.0 mg, 45.0 μmol) and (1H-indol-5-yl)boronic acid (10.9 mg, 67.5 μmol) afforded 3-((2-((2-(1H-indol-5-yl)benzo[d]thiazol-6-yl)amino)-6-methylquinazolin-4-yl)amino)propan-1-ol (70) (19.6 mg, 91%). ¹H NMR (400 MHZ, DMSO-d₆) δ 12.6 (br s, 1H), 11.5 (s, 1H), 10.6 (br s, 1H), 9.60 (br s, 1H), 8.43 (br s, 1H), 8.32 (d. J=1.7 Hz, 1H), 8.13 (s, 1H), 8.03 (d. J=8.7 Hz, 1H), 7.87 (dd, J=8.5, 1.8 Hz, 1H), 7.71-7.61 (m, 2H), 7.59-7.48 (m, 3H), 6.64-6.61 (m, 1H), 4.83-4.44 (m, 1H), 3.72-3.63 (m, 2H), 3.57-3.51 (m, 2H), 2.43 (s, 3H), 1.90-1.81 (m, 2H); HR-MS (ESI): Calcd for C₂₇H₂₈N₆OS⁺ [M+H]⁺; 481.1805; found, 481.1829.

General synthetic procedure for 71-82: A mixture of 3-((6-bromo-2-((2-methylbenzo[d]thiazol-6-yl)amino)quinazolin-4-yl)amino)propan-1-ol (D) (15.0 mg. 33.8 μmol, 1.0 equiv), N,N-Diisopropylethylamine (DIPEA; 35.4 μl, 203 μmol, 6.0 equiv) and amine (203 μmol, 6.0 equiv) in 3-pentanol (0.10 M) was heated in a microwave vial at 120° C. overnight. The reaction was cooled to room temperature, and product formation was confirmed by LCMS. The reaction mixture was then concentrated in vacuo and purified by column chromatography (Biotage SNAP cartridge, KP-NH, 11 g, 2%-30% MeOH in DCM) to afford compounds 71-82.

Synthesis of 71: Following general synthetic procedure 3 using D (15.0 mg, 33.8 μmol) and 1-benzylpiperidin-4-amine (38.6 mg, 203 μmol) afforded 3-((2-((2-((1-benzylpiperidin-4-yl)amino)benzo[d]thiazol-6-yl)amino)-6-methylquinazolin-4-yl)amino)propan-1-ol (71) (13.8 mg, 74%). ¹H NMR (400 MHZ, MeOD) δ 8.11 (d, J=1.9 Hz, 1H), 7.71-7.67 (m, 1H), 7.46-7.25 (m, 9H), 3.81-3.64 (m, 5H), 3.56 (s, 2H), 2.97-2.86 (m, 2H), 2.42 (s, 3H), 2.28-2.19 (m, 2H), 2.13-2.04 (m, 2H), 1.96-1.89 (m, 2H), 1.69-1.55 (m, 2H); HR-MS (ESI): Calcd for C₃₁H₃₆N₇OS⁺ [M+H]⁺; 554.2697; found, 554.2722.

Synthesis of 72: Following general synthetic procedure 3 using D (15.0 mg. 33.8 μmol) and morpholine (17.7 mg. 203 μmol) afforded 3-((6-methyl-2-((2-morpholinobenzo[d]thiazol-6-yl)amino)quinazolin-4-yl)amino)propan-1-ol (72) (7.3 mg, 48%). ¹H NMR (400 MHZ, MeOD) δ 8.26 (d. J=1.6 Hz, 1H), 7.71-7.67 (m, 1H), 7.48-7.40 (m, 3H), 7.33 (d, J=8.5 Hz, 1H), 3.84-3.79 (m, 4H), 3.74-3.64 (m, 4H), 3.60-3.54 (m, 4H), 2.42 (s, 3H), 2.00-1.89 (m, 2H) ¹³C NMR (150 MHz, DMSO-d₆) δ 167.3, 160.2, 157.0, 149.7, 146.5, 136.9, 134.5, 131.0, 130.7, 125.6, 122.4, 118.9, 118.3, 111.9, 110.6, 66.0 (2C), 59.2, 48.7 (2C), 38.4, 32.4, 21.3; HR-MS (ESI): Calcd for C₂₃H₂₇N₆O₂S⁺ [M+H]⁺; 451.1911; found, 451.1929.

Synthesis of 73: Following general synthetic procedure 3 using D (15.0 mg, 33.8 μmol) and 2-methylpropan-1-amine (14.8 mg, 203 μmol) afforded 3-((2-((2-(isobutylamino)benzo[d]thiazol-6-yl)amino)-6-methylquinazolin-4-yl)amino)propan-1-ol (73) (11.6 mg, 79%). ¹H NMR (400 MHZ, MeOD) δ 8.11 (d. J=2.0 Hz, 1H), 7.70-7.67 (m, 1H), 7.45-7.38 (m, 2H), 7.35-7.31 (m, 2H), 3.74-3.65 (m, 4H), 3.23 (d, J=7.0 Hz, 2H), 2.42 (s, 3H), 2.03-1.89 (m, 3H), 1.02 (s, 3H), 1.00 (s, 3H) ¹³C NMR (150 MHz, DMSO-d₆) δ 165.0, 160.2, 157.1, 149.8, 147.1, 136.2, 134.4, 130.7, 130.6, 125.6, 122.4, 117.9, 117.7, 111.8, 110.8, 59.3, 52.1, 38.4, 32.5, 28.2, 21.3, 20.6 (2C); HR-MS (ESI): Calcd for C₂₃H₂₇N₆OS⁻ [M−H]⁻; 435.1973; found, 435.1989.

Synthesis of 74: Following general synthetic procedure 3 using D (15.0 mg, 33.8 μmol) and 2-morpholinoethan-1-amine (26.4 mg. 203 μmol) afforded 3-((6-methyl-2-((2-((2-morpholinoethyl)amino)benzo[d]thiazol-6-yl)amino)quinazolin-4-yl)amino)propan-1-ol (74) (12.6 mg. 75%). ¹H NMR (400 MHZ, MeOD) δ 8.14-8.09 (m, 1H), 7.68 (br s, 1H), 7.45-7.29 (m, 4H), 3.77-3.63 (m, 8H), 3.57 (d, J=6.4 Hz, 2H), 2.66 (d. J=6.4 Hz, 2H), 2.60-2.49 (m, 4H), 2.41 (s, 3H), 1.98-1.88 (m, 2H), ¹³C NMR (150 MHz, DMSO-d₆) δ 164.8, 160.2, 157.1, 149.8, 147.0, 136.3, 134.4, 130.8, 130.6, 125.6, 122.4, 118.0, 117.7, 111.9, 110.8, 66.6 (2C), 59.3, 57.6, 53.8 (2C), 41.5, 38.4, 32.5, 21.3: HR-MS (ESI): Calcd for C₂₅H₃₂N₇O₂S⁺ [M+H]⁺; 494.2333; found, 494.2357.

Synthesis of 75: Following general synthetic procedure 3 using D (15.0 mg, 33.8 μmol) and benzo[d][1,3]dioxol-5-ylmethanamine (29.1 mg, 203 μmol) afforded 3-((2-((2-((benzo[d][1,3]dioxol-5-ylmethyl)amino)benzo[d]thiazol-6-yl)amino)-6-methylquinazolin-4-yl)amino)propan-1-ol (75) (8.9 mg, 51%). ¹H NMR (400 MHZ, MeOD) δ 7.89 (s, 1H), 7.83 (d, J=2.0 Hz, 1H), 7.60 (dd, J=8.5, 1.4 Hz, 1H), 7.48 (d, J=8.5 Hz, 1H), 7.40 (d, J=8.5 Hz, 1H), 7.37-7.32 (m, 1H), 6.91-6.86 (m, 2H), 6.79 (d, J=11.8 Hz, 1H), 5.93 (s, 2H), 4.54 (s, 2H), 3.74 (t. J=7.1 Hz, 2H), 3.65 (t, J=6.1 Hz, 2H), 2.46 (s, 3H), 1.98-1.88 (m, 2H); HR-MS (ESI): Calcd for C₂₇H₂₇N₆O₃S⁺ [M+H]⁺; 515.1860; found, 515.1883.

Synthesis of 76: Following general synthetic procedure 3 using D (15.0 mg, 33.8 μmol) and 2-phenoxyethan-1-amine (27.8 mg, 203 μmol) afforded 3-((6-methyl-2-((2-((2-phenoxyethyl)amino)benzo[d]thiazol-6-yl)amino)quinazolin-4-yl)amino)propan-1-ol (76) (6.3 mg, 37%). ¹H NMR (400 MHZ, MeOD) δ 7.95-7.89 (m, 2H), 7.68-7.63 (m, 1H), 7.56-7.51 (m. 1H), 7.48-7.41 (m, 2H), 7.31-7.24 (m, 2H), 6.99-6.90 (m, 3H), 4.25 (t, J=5.2 Hz, 2H), 3.90 (t, J=5.2 Hz, 2H), 3.76 (t, J=7.1 Hz, 2H), 3.65 (t. J=6.1 Hz, 2H), 2.47 (s, 3H), 1.98-1.88 (m, 2H); HR-MS (ESI): Calcd for C₂₇H₂₇N₆O₃S⁺ [M+H]⁺; 515.1860; found, 515.1883.

Synthesis of 77: Following general synthetic procedure 3 using D (15.0 mg, 33.8 μmol) and 2-(benzyloxy)ethan-1-amine (30.7 mg, 203 μmol) afforded 3-((2-((2-((2-(benzyloxy)ethyl)amino)benzo[d]thiazol-6-yl)amino)-6-methylquinazolin-4-yl)amino)propan-1-ol (77) (7.7 mg, 44%). ¹H NMR (400 MHZ, MeOD) δ 7.98-7.91 (m, 2H), 7.68-7.63 (m, 1H), 7.52-7.46 (m, 2H), 7.44 (d, J=8.5 Hz, 1H), 7.36-7.21 (m, 5H), 4.59 (s, 2H), 3.79-3.70 (m, 6H), 3.65 (t. J=6.1 Hz, 2H), 2.48 (s, 3H), 1.98-1.88 (m, 2H); HR-MS (ESI): Calcd for C₂₈H₃₁N₆O₂S⁺ [M+H]⁺; 515.2224; found, 515.2246.

Synthesis of 78: Following general synthetic procedure 3 using D (15.0 mg, 33.8 μmol) and 1-amino-2-methylpropan-2-ol (18.1 mg. 203 μmol) afforded 3-((2-((2-((2-hydroxy-2-methylpropyl)amino)benzo[d]thiazol-6-yl)amino)-6-methylquinazolin-4-yl)amino)propan-1-ol (78) (14.0 mg, 91%). ¹H NMR (400 MHZ, MeOD) δ 8.12 (d, J=2.1 Hz, 1H), 7.68 (br s, 1H), 7.44-7.37 (m, 2H), 7.35-7.30 (m, 2H), 3.73-3.64 (m, 4H), 3.44 (s, 2H), 2.41 (s, 3H), 1.98-1.87 (m, 2H), 1.27 (s, 6H); HR-MS (ESI): Calcd for C₂₃H₂₉N₆O₂S⁺ [M+H]⁺; 453.2067; found, 453.2069.

Synthesis of 79: Following general synthetic procedure 3 using D (15.0 mg, 33.8 μmol) and N1,N1-dimethylethane-1,2-diamine (17.9 mg, 203 μmol) afforded 3-((2-((2-((2-(dimethylamino)ethyl)amino)benzo[d]thiazol-6-yl)amino)-6-methylquinazolin-4-yl)amino)propan-1-ol (79) (12.9 mg, 84%). ¹H NMR (400 MHZ, MeOD) δ 8.14 (d, J=2.0 Hz, 1H), 7.68 (br s, 1H), 7.44-7.38 (m, 2H), 7.38-7.30 (m, 2H), 3.73-3.65 (m, 4H), 3.56 (t, J=6.7 Hz, 2H), 2.64 (t, J=6.7 Hz, 2H), 2.42 (s, 3H), 2.32 (s, 6H), 1.98-1.89 (m, 2H); HR-MS (ESI): Calcd for C₂₃H₃₀N₇OS⁺ [M+H]⁺; 452.2227; found, 452.2242.

Synthesis of 80: Following general synthetic procedure 3 using D (15.0 mg, 33.8 μmol) and 2,2′-azanediylbis(ethan-1-ol) (21.3 mg. 203 μmol) afforded 2,2′-((6-((4-((3-hydroxypropyl)amino)-6-methylquinazolin-2-yl)amino)benzo[d]thiazol-2-yl)azanediyl)bis(ethan-1-ol) (80) (5.2 mg, 33%). ¹H NMR (400 MHZ, MeOD) δ 8.10-8.02 (m, 1H), 7.95-7.90 (m, 1H), 7.65 (dd, J=8.5, 1.3 Hz, 1H), 7.56-7.50 (m, 2H), 7.44 (d, J=8.5 Hz, 1H), 3.95-3.88 (m, 4H), 3.88-3.82 (m, 4H), 3.76 (t, J=7.2 Hz, 2H), 3.66 (t, J=6.1 Hz, 2H), 2.47 (s, 3H), 1.98-1.88 (m, 2H); HR-MS (ESI): Calcd for C₂₃H₂₉N₆O₃S⁺ [M+H]⁺; 469.2016; found, 469.2031.

Synthesis of 81: Following general synthetic procedure 3 using D (15.0 mg, 33.8 μmol) and 4-(2-aminoethyl)benzenesulfonamide (40.7 mg, 203 μmol) afforded 4-(2-((6-((4-((3-hydroxypropyl)amino)-6-methylquinazolin-2-yl)amino)benzo[d]thiazol-2-yl)amino)ethyl)benzenesulfonamide (81) (7.5 mg, 39%). ¹H NMR (400 MHZ, MeOD) δ 7.94-7.91 (m. 1H), 7.91-7.82 (m, 3H), 7.67 (dd, J=8.5, 1.4 Hz, 1H), 7.53-7.40 (m, 5H), 3.82-3.71 (m. 4H), 3.65 (t. J=6.1 Hz, 2H), 3.11 (t. J=7.0 Hz, 2H), 2.48 (s, 3H), 1.99-1.89 (m, 2H); HR-MS (ESI): Calcd for C₂₇H₃₀N₇O₃S₂⁺ [M+H]⁺; 564.1846; found, 564.1856.

Synthesis of 82: Following general synthetic procedure 3 using D (15.0 mg, 33.8 μmol) and 1-methylpiperazine (20.3 mg, 203 μmol) afforded 3-((6-methyl-2-((2-(4-methylpiperazin-1-yl)benzo[d]thiazol-6-yl)amino)quinazolin-4-yl)amino)propan-1-ol (82) (2.9 mg, 18%). ¹H NMR (400 MHZ, MeOD) δ 8.25 (d. J=1.7 Hz, 1H), 7.69 (br s. 1H), 7.47-7.39 (m, 3H), 7.37-7.30 (m, 1H), 3.75-3.58 (m, 8H), 2.65 (t. J=10.2 Hz, 4H), 2.42 (s, 3H), 2.36 (s, 3H), 1.98-1.88 (m, 2H); HR-MS (ESI): Calcd for C₂₄H₃₀N₇OS⁺ [M+H]⁺; 464.2227; found, 464.2235.

### High-Throughput Screen of the RNA-Focused Small Molecule Library

In vitro IC₅₀ measurements: IC₅₀ measurements for disruption of r(CCUG)₁₂-MBNL1 complex was completed using a previously reported TR-FRET assay with minor modifications.^{23, 24} Briefly, 5′-biotinylated r(CCUG)₁₂ (SEQ ID NO: 15) was folded in 1× Folding Buffer (20 mM HEPES, pH 7.5, 110 mM KCl, and 10 mM NaCl) at 60° C. for 5 min then cooled to room temperature. The buffer was adjusted to 1× Assay Buffer (20 mM HEPES, pH 7.5, 110 mM KCl, 10 mM NaCl, 2 mM MgCl₂, 2 mM CaCl₂), 5 mM DTT. 0.1% BSA, and 0.5% Tween-20).

Next, MBNL1-His6 was added, and the samples were incubated at room temperature for 15 min. Then compounds were added, and the samples were incubated for another 15 min at room temperature. The final concentrations of r(CCUG)₁₂ (SEQ ID NO: 15) and MBNL1 were 80 nM and 60 nM. A solution of streptavidin-XL665 and anti-His6-Tb antibody was then added in a total volume of 10 μL, with final concentrations of 40 nM and 0.44 ng/μL, respectively. The samples were incubated for 30 min at room temperature and added to a white 384-well plate, where TR-FRET was measured on a Molecular Devices SpectraMax M5 plate reader using an excitation wavelength of 345-nm and a 420-nm cutoff. The ratios of fluorescence intensity at 545 and 665 nm were calculated, and ratios in the absence of a compound and in the absence of RNA were used to calculate percent disruption. The resulting curves were fit to equation 1 to determine IC₅₀ values:

$$y = B + \frac{A - B}{1 + {( \frac{IC 50}{x} )}^{hillslope}} \qquad ( Eq . 1 )$$

where y is ratio of fluorescence intensities at 545 nm and 665 nm (F545/F665), x is the concentration of a compound, B is F545/F665 value at max FRET effect (solution has RNA and protein but no compound added); A is F545/F665 value at min FRET effect (solution has antibodies but no RNA, protein, or compound); and the IC₅₀ is the concentration of a compound where half of the protein is displaced by a compound.

NMR samples preparation: A self-complementary RNA construct, r(5′-GACAGCCUGCUGUC-3′), SEQ ID NO:12, was purchased from GE Healthcare Dharmacon, Inc., deprotected according to the manufacturer's recommended protocol, and desalted with PD-10 columns (GE Healthcare, cat: 17-0851-01) also per the manufacturer's protocol. RNA samples were dissolved in NMR Buffer [10 mM KH₂PO₄/K₂HPO₄ and 0.05 mM EDTA (pH 6.0)] and folded by heating to 95° C. for 3 min and slowly cooling to room temperature.

NMR spectroscopy: NMR spectra were acquired at 25° C. on Bruker Avance III 600 and 700 MHZ spectrometers equipped with cryoprobes. 1D NMR spectra were acquired on samples containing 100 μM of RNA alone in 100% D20. Compounds 38, 40, 41, and 42 were each titrated into separate samples at 0.5, 1.0, 1.5, and 2.0 compound: RNA molar ratios. WaterLOGSY (water-ligand observed via gradient spectroscopy) spectra²⁹ were acquired on samples containing 300 μM of each compound alone or in the presence of 15 μM of RNA at a 20:1 compound:RNA ratio in H₂O, to which D₂O was added to 5% by volume. WaterLOGSY spectra were phased to give negative NOEs for non-binders. 2D NOESY and DQF-COSY spectra were acquired on samples containing 400 μM of RNA alone in 100% D₂O. 2D NMR spectra were processed with nmrPipe³⁰ and assigned with SPARKY.³¹

Cell lines and cell culture: Compounds were tested in two cell lines: (i) DM2 patient-derived fibroblasts (generous gift from University of Florida, Center for NeuroGenetics) and (ii) fibroblasts from a healthy donor (wild-type; WT GM07492; Coriell Institute). Cells were maintained at 37° ° C. with 5% CO₂. DM2 fibroblasts were cultured in DMEM/high glucose (HyClone) supplemented with 20% FBS (Sigma) and 1% Antibiotic-Antimycotic solution (Corning). Wild-type fibroblasts were cultured in MEM (Corning) supplemented with 10% FBS, 1% Glutagro (Corning), and 1% Antibiotic-Antimycotic solution.

Evaluation of cell viability: Compound toxicity in DM2 fibroblasts was evaluated using a CellTiter-Glo Kit (Promega) as described in the manufacturer's protocol. Briefly, after 48 h treatment in 96-wells plates, 100 μL of the CellTiter-Glo reagent was added to each well. The plate was then incubated at room temperature for 10 min, and luminescence was measured using BioTek FLX-800 luminescence plate reader (n=6 replicates; 1 independent experiment).

Evaluation of pre-mRNA splicing via RT-PCR: Pre-mRNA splicing was analyzed as previously described.¹⁰ Briefly, cells were grown in 6-well plates and treated with the desired compound in growth medium at ˜40% confluency. After 48 h, the cells were lysed, and total RNA was harvested using a Zymo Quick RNA Miniprep Kit. Approximately 1 μg of total RNA was reverse transcribed using a qScript cDNA synthesis kit (20 μL of total reaction volume, Quanta BioSciences); 2 μL of the RT reaction was used for PCR using GoTaq DNA polymerase (Promega). RT-PCR products were observed after 35 cycles of 95° C. for 30 s, 58° C. for 30s, 72 ºC for 1 min, and a final extension at 72° C. for 5 min. Products were separated on a 2% agarose gel (110 V for 1 h in 13TBE buffer), visualized by staining with ethidium bromide, and imaged using a Typhoon 9410 variable mode imager. Gels were quantified using ImageJ. Percent rescue was calculated by dividing the difference between treated and untreated DM2 samples by the difference between untreated DM2 and WT samples (Equation 2).

$$\% Rescue = \frac{\% exon exclusion DM 2 - \% exon exclusion treated}{\% exon exclusion DM 2 - \% exon exclusion WT} * 100 \qquad ( Eq . 2 )$$

Evaluation of CNBP abundance via RT-qPCR: CNBP abundance was evaluated as previously described.¹⁰ Briefly, cells were grown in 6-well plates and treated with the desired compound in growth medium at 40% confluency. After 48 h, the cells were lysed, and total RNA was harvested using a Zymo Quick RNA Miniprep Kit per the manufacturer's protocol. Approximately 1 μg of total RNA was reverse transcribed using a qScript cDNA synthesis kit (20 μL total reaction volume, Quanta BioSciences); 2 μL of the reverse transcription (RT) reaction was used for each primer pair (Table S1) for quantitative PCR with SYBR Green Master Mix, performed on a QuantStudio 5, 384-well Block Real-Time PCR System (Applied Biosciences). Relative abundance was determined by normalizing to GAPDH (n=6 replicates per concentration; 2 independent experiments).

Evaluation of nuclear foci via RNA fluorescence in situ hybridization (FISH): RNA FISH was used to determine the small molecules' effects on the number of nuclear foci as previously described.¹⁰ The number of foci were counted in 40 nuclei/replicate (120 total nuclei counted); n=3 replicates; 1 independent experiment.

Affinity measurements: Binding affinity measurements were performed via microscale thermophoresis (MST) on a Monolith NT.115 system (NanoTemperTechnologies) with Cy5-labeled (CCUG)₁₂ (SEQ ID NO: 15) (5′-Cy5-GCG(CCUG)₁₂CGC. SEQ ID NO:13; Dharmacon) and Cy5-labeled base pair control (BP; 5′-Cy5-GCG(CCUG)(GCAG)₅CGC, SEQ ID NO:14; Dharmacon), which were deprotected according to the manufacturer's protocol and then desalted using a PD-10 column (GE LifeSciences) per the manufacturer's recommended procedure. RNA (5 nM) was prepared in 8 mM Na₂HPO₄, 185 mM NaCl, 1 mM EDTA and folded by heating at 60° C. for 5 min and slowly cooling to room temperature. Compound 1:1 dilution was prepared using 1×MST Buffer (8 mM Na₂HPO₄, 185 mM NaCl, 1 mM EDTA) and 0.1% (v/v) Tween-20 was added. After cooling the RNA solution was added to the compound solution (0.05% (v/v) Tween-20 final concentration). Samples were incubated for 30 min at room temperature and then loaded into standard capillaries (NanoTemper Technologies). The following parameters were used to acquire thermophoretic data: 5-20% LED, 80% MST power, Laser-On time=30 s, Laser-Off time=5 s. Fluorescence was detected using excitation wavelengths of 605-645 nm and emission wavelengths of 680-685 nm. The resulting data were analyzed by thermophoresis analysis and fitted using a quadratic binding equation in the MST analysis software (NanoTemper Technologies). Dissociation constants were then determined using equation 3. The reported K_{d} values are an average of two independent sets of experiments. Eq. 3:

$${K}_{d} = \frac{unbo u n d + ( b o u n d - u n b o u n d )}{2} * ( [ R N A ] + [ 2 b ] + {K}_{d} \sqrt{{( [ R N A ] + [ 2 b ] + {K}_{d} )}^{2} - 4 ( [ RNA \vert ] * [ 2 b ] )}$$

TABLE S1
Sequences of primers used for RT-PCR and RT-qPCR

| | Forward Primer | | Reverse Primer | | |
|---|---|---|---|---|---|
| Gene | (5′-3′) | | (5′-3′) | | Purpose |
| MAP4K4 | CCTCATCCAGTGAGGAG | SEQ | TGGTGGGAGA | SEQ ID | RT-PCR |
| Exon 22a | TCG | ID | AATGCTGTATG | NO: 7 | |
| | | NO: 2 | C | | |
| IR Exon 11 | CCAAAGACAGACTCTCA | SEQ | AACATCGCCA | SEQ ID | RT-PCR |
| | GAT | ID | AGGGACCTGC | NO: 8 | |
| | | NO: 3 | | | |
| GAPDH | AAGGTGAAGGTCGGAGT | SEQ | AATGAAGGGG | SEQ ID | qPCR |
| | CAA | ID | TCATTGATGG | NO: 9 | |
| | | NO: 4 | | | |
| CNBP intron 1 | ATTCCAAGGTTGGTTGA | SEQ | AACCCAAACC | SEQ ID | qPCR |
| | AGC | ID | AATGAAGCTG | NO: 10 | |
| | | NO: 5 | | | |
| CNBP mature | AAACTGGTCATGTAGCC | SEQ | AATTGTGCATT | SEQ ID | qPCR |
| mRNA | ATCAAC | ID | CCCGTGCAAG | NO: 11 | |
| | | NO: 6 | | | |

### REFERENCES

1. Paulson, H., Repeat expansion diseases. Handb. Clin. Neurol. 2018, 147, 105-123.
2. Hale, M. A.; Johnson, N. E.; Berglund, J. A., Repeat-associated RNA structure and aberrant splicing. Biochim. Biophys. Acta, Gene Regul. Mech. 2019, 1862 (11-12), 194405.
3. Cleary, J. D.; Pattamatta, A.; Ranum, L. P. W., Repeat-associated non-ATG (RAN) translation. J. Biol. Chem. 2018, 293 (42), 16127-16141.
4. Sznajder, Ł. J.; Thomas, J. D.; Carrell, E. M.; Reid, T.; McFarland, K. N.; Cleary, J. D.; Oliveira, R.; Nutter, C. A.; Bhatt, K.; Sobczak, K., et al., Intron retention induced by microsatellite expansions as a disease biomarker. Proc. Natl. Acad. Sci. U.S.A 2018, 115 (16), 4234.
5. Liquori, C. L.; Ricker, K.; Moseley, M. L.; Jacobsen, J. F.; Kress, W.; Naylor, S. L.; Day, J. W.; Ranum, L. P. W., Myotonic Dystrophy Type 2 Caused by a CCTG Expansion in Intron 1 of ZNF9. Science (New York, N.Y.) 2001, 293 (5531), 864.
6. Childs-Disney, J. L.; Yildirim, I.; Park, H.; Lohman, J. R.; Guan, L.; Tran, T.; Sarkar, P.; Schatz, G. C.; Disney, M. D., Structure of the Myotonic Dystrophy Type 2 RNA and Designed Small Molecules That Reduce Toxicity. ACS Chem. Biol. 2014, 9 (2), 538-550.
7. Fardaei, M.; Rogers, M. T.; Thorpe, H. M.; Larkin, K.; Hamshere, M. G.; Harper, P. S.; Brook, J. D., Three proteins, MBNL, MBLL and MBXL, co-localize in vivo with nuclear foci of expanded-repeat transcripts in DM1 and DM2 cells. Hum. Mol. Genet. 2002, 11 (7), 805-14.
8. Mankodi, A.; Urbinati, C. R.; Yuan, Q. P.; Moxley, R. T.; Sansone, V.; Krym, M.; Henderson, D.; Schalling, M.; Swanson, M. S.; Thornton, C. A., Muscleblind localizes to nuclear foci of aberrant RNA in myotonic dystrophy types 1 and 2. Hum. Mol. Genet. 2001, 10 (19), 2165-70.
9. Savkur, R. S.; Philips, A. V.; Cooper. T. A., Aberrant regulation of insulin receptor alternative splicing is associated with insulin resistance in myotonic dystrophy. Nat. Genet. 2001, 29 (1), 40-7.
10. Benhamou, R. I.; Angelbello, A. J.; Wang, E. T.; Disney, M. D., A Toxic RNA Catalyzes the Cellular Synthesis of Its Own Inhibitor, Shunting It to Endogenous Decay Pathways. Cell Chem. Biol. 2020, 27 (2), 223-231.e4.
11. Wieben, E. D.; Aleff, R. A.; Tosakulwong, N.; Butz, M. L.; Highsmith, W. E.; Edwards, A. O.; Baratz, K. H., A common trinucleotide repeat expansion within the transcription factor 4 (TCF4, E2-2) gene predicts Fuchs corneal dystrophy. PloS one 2012, 7 (11), e49083-e49083.
12. Angelbello, A. J.; Benhamou, R. I.; Rzuczek, S. G.; Choudhary, S.; Tang. Z.; Chen, J. L.; Roy. M.; Wang, K. W.; Yildirim, I.; Jun, A. S., et al., A small molecule that binds an RNA repeat expansion stimulates its decay via the exosome complex. Cell Chem. Biol. 2020, doi.org/10.1016/j.chembiol. 2020.10.007.
13. Angelbello, A. J.; Rzuczek, S. G.; Mckee, K. K.; Chen, J. L.; Olafson, H.; Cameron, M. D.; Moss, W. N.; Wang, E. T.; Disney, M. D., Precise small-molecule cleavage of an r(CUG) repeat expansion in a myotonic dystrophy mouse model. Proc. Natl. Acad. Sci. U.S.A 2019, 116 (16), 7799-7804.
14. Costales, M. G.; Aikawa, H.; Li, Y.; Childs-Disney, J. L.; Abegg. D.; Hoch, D. G.; Pradeep Velagapudi, S.; Nakai, Y.; Khan, T.; Wang, K. W., et al., Small-molecule targeted recruitment of a nuclease to cleave an oncogenic RNA in a mouse model of metastatic cancer. Proc. Natl. Acad. Sci. U.S.A 2020, 117 (5), 2406-2411.
15. Disney, M. D., Targeting RNA with Small Molecules To Capture Opportunities at the Intersection of Chemistry, Biology, and Medicine. J. Am. Chem. Soc. 2019, 141 (17), 6776-6790.
16. Benhamou, R. I.; Angelbello, A. J.; Andrews, R. J.; Wang, E. T.; Moss, W. N.; Disney, M. D., Structure-Specific Cleavage of an RNA Repeat Expansion with a Dimeric Small Molecule Is Advantageous over Sequence-Specific Recognition by an Oligonucleotide. ACS Chem. Biol. 2020, 15 (2), 485-493.
17. Benhamou, R. I.; Abe, M.; Choudhary, S.; Meyer, S. M.; Angelbello, A. J.; Disney, M. D., Optimization of the Linker Domain in a Dimeric Compound that Degrades an r(CUG) Repeat Expansion in Cells. J. Med. Chem. 2020, 63 (14), 7827-7839.
18. Wong, C.-H.; Fu, Y.; Ramisetty, S. R.; Baranger, A. M.; Zimmerman, S. C., Selective inhibition of MBNL1-CCUG interaction by small molecules toward potential therapeutic agents for myotonic dystrophy type 2 (DM2). Nucleic Acids Res. 2011, 39 (20), 8881-8890.
19. Haniff, H. S.; Graves, A.; Disney, M. D., Selective Small Molecule Recognition of RNA Base Pairs. ACS Comb. Sci. 2018, 20 (8), 482-491.
20. Lipinski, C. A.; Lombardo, F.; Dominy, B. W.; Feeney, P. J., Experimental and computational approaches to estimate solubility and permeability in drug discovery and development settings. Adv. Drug Delivery Rev. 1997, 23 (1), 3-25.
21. Shultz, M. D., Two Decades under the Influence of the Rule of Five and the Changing Properties of Approved Oral Drugs. J. Med. Chem. 2019, 62 (4), 1701-1714.
22. DeGoey, D. A.; Chen, H.-J.; Cox, P. B.; Wendt, M. D., Beyond the Rule of 5: Lessons Learned from Abb Vie's Drugs and Compound Collection. J. Med. Chem. 2018, 61 (7), 2636-2651.
23. Costales, M. G.; Rzuczek, S. G.; Disney, M. D., Comparison of small molecules and oligonucleotides that target a toxic, non-coding RNA. Bioorg. Med. Chem. Lett. 2016, 26 (11), 2605-2609.
24. Chen, C. Z.; Sobczak, K.; Hoskins, J.; Southall, N.; Marugan, J. J.; Zheng, W.; Thornton, C. A.; Austin, C. P., Two High-Throughput Screening Assays for Aberrant RNA-Protein Interactions in Myotonic Dystrophy Type 1. Anal. Bioanal. Chem. 2012, 402 (5), 1889-1898.
25. Ule, J.; Ule, A.; Spencer, J.; Williams, A.; Hu, J. S.; Cline, M.; Wang, H.; Clark, T.; Fraser, C.; Ruggiu, M., et al., Nova regulates brain-specific splicing to shape the synapse. Nat. Genet. 2005, 37 (8), 844-52.
26. Angelbello, A. J.; DeFeo, M. E.; Glinkerman, C. M.; Boger, D. L.; Disney, M. D., Precise Targeted Cleavage of a r(CUG) Repeat Expansion in Cells by Using a Small-Molecule-Deglycobleomycin Conjugate. ACS Chem. Biol. 2020, 15 (4), 849-855.
27. Haniff, H. S.; Tong, Y.; Liu, X.; Chen, J. L.; Suresh, B. M.; Andrews, R. J.; Peterson, J. M.; O'Leary, C. A.; Benhamou, R. I.; Moss, W. N., et al., Targeting the SARS-COV-2 RNA Genome with Small Molecule Binders and Ribonuclease Targeting Chimera (RIBOTAC) Degraders. ACS Cent. Sci. 2020, 6 (10), 1713-1721.
28. DeJesus-Hernandez, M.; Mackenzie, I. R.; Boeve, B. F.; Boxer, A. L.; Baker, M.; Rutherford, N. J.; Nicholson, A. M.; Finch, N. A.; Flynn, H.; Adamson, J., et al., Expanded GGGGCC hexanucleotide repeat in noncoding region of C9ORF72 causes chromosome 9p-linked FTD and ALS. Neuron 2011, 72 (2), 245-56.
29. Dalvit, C.; Pevarello, P.; Tato, M.; Veronesi, M.; Vulpetti, A.; Sundstrom, M., Identification of compounds with binding affinity to proteins via magnetization transfer from bulk water. J. Biomol. NMR 2000, 18 (1), 65-8.
30. Delaglio, F.; Grzesiek, S.; Vuister, G. W.; Zhu, G.; Pfeifer, J.; Bax, A., NMRPipe: A multidimensional spectral processing system based on UNIX pipes. J. Biomol. NMR 1995, 6 (3), 277-293.
31. Goddard, T. D.; Kneller, D. G. SPARKY, NMR Assignment and Integration Software, 3; University of California, San Francisco: San Francisco, 2004.
32. Chen, C. Z.; Sobczak, K.; Hoskins, J.; Southall, N.; Marugan, J. J.; Zheng. W.; Thornton, C. A.; Austin, C. P., Two high-throughput screening assays for aberrant RNA-protein interactions in myotonic dystrophy type 1. Anal. Bioanal. Chem. 2012, 402 (5), 1889-98.

### MISCELLANEOUS STATEMENTS

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The inventions, examples, biological assays and results described and claimed herein have may attributes and embodiments include, but not limited to, those set forth or described or referenced in this application.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

Unless defined otherwise, all technical and scientific terms used herein have the same meaning as commonly understood by a person of ordinary skill in the art.

What is claimed is:

1. A composition comprising a quinazoline compound of Formula I 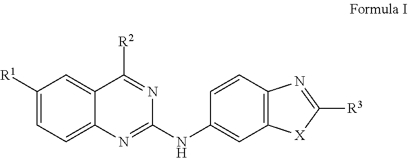
wherein
R¹ is hydrogen or alkyl of 1 to 3 carbons;
R² is —NH(CH₂)ₙOH with n being an integer of 2, 3 or 4
R³ is hydrogen, alkyl of 1 to 3 carbons, —NH(CH₂)C(R⁴)₂OH with each instance of R⁴ independently being hydrogen or methyl or —NH(CH₂)₂ NMe₂;
X is oxygen, sulfur or NH.

2. A composition according to claim 1 wherein X is sulfur.

3. A composition according to claim 1 wherein X is oxygen.

4. A composition according to claim 1 wherein X is NH.

5. A composition according to any of claims 1-4 wherein R¹ is alkyl.

6. A composition according to claim 5 wherein R¹ is methyl.

7. A composition according to any of claims 1-6 wherein n is 2 or 3.

8. A composition according to claim 7 wherein n is 3.

9. A composition according to any of claims 1-8 wherein R³ is alkyl.

10. A composition according to claim 9 wherein R³ is methyl.

11. A composition according to any of claims 1-8 wherein R³ is —NH(CH₂)C(R⁴)₂OH.

12. A composition according to claim 11 wherein at least one R⁴ is methyl.

13. A composition according to claim 11 wherein both R⁴'s are methyl.

14. A composition according to any of claims 1-8 wherein R³ is —NH(CH₂)₂ NMe₂.

15. A composition according to claim 1 wherein X is sulfur, R¹ is methyl, R² is —NH(CH₂)₃OH, and R³ is methyl.

16. A composition according to claim 1 wherein X is sulfur, R¹ is methyl, R² is —NH(CH₂)₃OH, and R³ is —NH(CH₂)C(CH₃)₂OH.

17. A complex of r(CCUG)^{exp} and a composition of claim 15 having a K_{d} in the range of 170-220 nM.

18. A method for reducing r(CCUG)^{exp}-MBNL1 complexation comprising contacting a composition of any of claims 1-16 with DM2 patient derived fibroblasts carrying r(CCUG)^{exp}-MBNL1 nuclear foci and observing a reduction of the number of r(CCUG)^{exp}-MBNL1 complexes by assaying for uncomplexed MBNL1 and/or uncomplexed r(CCUG)^{exp}, which are/is free of the nuclear foci.

19. A method according to claim 18 wherein the presence of uncomplexed MBNL1 is determined by immunofluorescence.

20. A pharmaceutical composition comprising a composition of any of claims 1-16 and a pharmaceutically acceptable carrier.

21. A pharmaceutical composition according to claim 20 comprising a composition of claim 15 and a pharmaceutical carrier.

22. A method for reducing the abundance of the repeat expansion and incidence of foci in myotonic dystrophy type 2 cells comprising contacting a tissue sample of a DM2 patient carrying DM type 2 cells with a pharmaceutical composition of claim 20.

23. A method according to claim 22 wherein an effective amount of a composition of claim 21 is administered.

24. A method for treating myotonic dystrophy type 2 in a patient comprising administering to the patient an effective amount of a composition of any of claims 1-16.

25. A method for treating a disease caused by or associated with r(CCCUG)^{exp} in a patient comprising administering to the patient an effective amount of a composition of any of claims 1-16.

26. A method according to claim 24 or 25 wherein an effective amount of a composition of claim 15 is administered.

27. A method for treating myotonic dystrophy type 2 in a patient comprising administering to the patient an effective amount of a pharmaceutical composition of claim 20.

28. A method for treating a disease caused by or associated with r(CCCUG)^{exp} in a patient comprising administering to the patient an effective amount of a pharmaceutical composition of claim 20.

29. A method according to claim 27 or 28 wherein the pharmaceutical composition of claim 20 comprises a composition of claim 15.

30. A method to rescue formation of r(CCUG)^{exp}-MBNL1 foci comprising contacting the foci with a quinazoline compound of any of claims 1-16 and visualizing uncomplexed MBNL1 by immunohistochemistry and/or uncomplexed r(CCUG)^{exp} by RNA fluorescence in situ hybridization (FISH).

31. A method for rescuing aberrant alternative pre-mRNA splicing in fibroblasts derived from a patient with aberrant alternative mRNA caused by r(CCUG)^{exp} comprising contacting the fibroblasts with a quinazoline compound of any of claims 1-16 and observing a rescue of alternative splicing defects.

32. A method according to claim 30 wherein the rescue of alternative mRNA splicing defects is determined by RT-qPCR or RNA-seq.

33. A composition according to any of claims 1-16 further comprising a combination with an r(CCUG)₁₂-MBNL1 complex.

34. A composition comprising second quinazoline compound of Formula II in combination with an r(CCUG)₁₂-MBNL1 complex or a single 2×2 CU/UC RNA 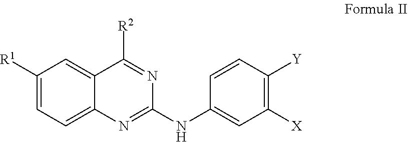
wherein
R¹ is hydrogen or alkyl of 1 to 3 carbons;
R² is —NH(CH₂)ₙOH with n being an integer of 2, 3 or 4, —NHCH₂-(2-tetrahydrofuranyl);
Y is —OMe, —NMe₂, —NHAc and X is H or Y and X together are —O—CH₂—CH₂—O—.

35. A composition according to claim 34 wherein R¹ is hydrogen.

36. A composition according to claim 34 wherein R¹ is methyl.

37. A composition according to claim 35 or 36 wherein R² is NH(CH₂)₃OH.

38. A composition according to any of claims 34-37 wherein Y and X together are —O—CH₂—CH₂—O—.

39. A method for alleviating DM2 defects resulting from r(CCUG)^{exp}-MBNL1 complexation by interrupting the complexation comprising contacting DM2 patient derived fibroblasts carrying r(CCUG)^{exp}-MBNL1 nuclear foci with a second quinazoline compound of any of claims 34-38.

40. A method according to claim 39 wherein alleviation is demonstrated by observing a reduction of the number of r(CCUG)^{exp}-MBNL1 complexes by detecting the presence of uncomplexed MBNL1 and/or uncomplexed r(CCUG)^{exp}.

41. A method according to claim 40 wherein the presence of uncomplexed MBNL1 is determined by immunofluorescence and/or uncomplexed r(CCUG)^{exp} is determined by RNA FISH.

42. A method for rescuing aberrant alternative pre-mRNA splicing caused by r(CCUG)^{exp}, comprising contacting fibroblasts derived from a patient with aberrant alternative mRNA splicing with a second quinazoline compound of any of claims 34-38 and observing a rescue of alternative splicing defects.

43. A method according to claim 42 wherein the rescue of alternative mRNA splicing defects is determined by RT-qPCR or RNA-seq.
